(19) United States
(12) Patent Application Publication
(10) Pub. No.: US 2026/0204408 A1
(43) Pub. Date: Jul. 16, 2026

(54) SYSTEM, METHOD, AND APPARATUS FOR MOISTURE EVENT DETECTION

(71) Applicant: EtectRx, LLC, Gainesville, FL (US)

(72) Inventors: Adam Standley, Chapel Hill, NC (US); D Eric Buffkin, Gainesville, FL (US); Tony Christopher Carnes, Gainesville, FL (US); Brian Peace, Gainesville, FL (US); Nathan Tiu, Gainesville, FL (US)

(73) Assignee: EtectRx, LLC, Gainesville, FL (US)

(21) Appl. No.: 19/234,085

(22) Filed: Jun. 10, 2025

Related U.S. Application Data
(60) Provisional application No. 63/658,334, filed on Jun. 10, 2024.

(51) Int. Cl. G16H 40/63; G16H 40/20

(52) U.S. Cl. CPC G16H 40/63; G16H 40/20

(57) ABSTRACT

The present disclosure provides a system for tracking hygiene products, comprising a plurality of hygiene products, each hygiene product having an associated tag, a reader configured to detect the tags associated with the hygiene products, and a point of care unit configured to receive tracking information from the reader. The point of care unit is further configured to track any of the time since a hygiene product was last replaced, moisture events, and rounding checks.

### CROSS-REFERENCE TO RELATED APPLICATIONS

This application claims priority to U.S. Application No. 63/658,334, titled SYSTEM, METHOD, AND APPARATUS FOR MOISTURE EVENT DETECTION, filed Jun. 10, 2024, which is hereby incorporated by reference in its entirety.

### FIELD OF THE DISCLOSURE

The present disclosure relates generally to electronic systems, methods, and apparatuses for monitoring moisture in healthcare applications such as incontinence care, wound dressings, ostomies, ports, clothing, bedding, or other areas of interest where indication of moisture presence is desired.

### BACKGROUND

Healthcare facilities managing incontinence care face significant operational challenges. As individuals age or experience health complications, they may lose bladder control, mobility to access restrooms, or cognitive awareness of their toileting needs, resulting in widespread use of disposable sanitary products such as briefs, diapers, and absorbent pads.

Traditional incontinence management relies on scheduled manual checks by healthcare staff to determine when sanitary products require changing. Staff members inspect residents at predetermined intervals to assess whether products have been soiled. This manual approach presents several operational difficulties: staffing shortages and heavy workloads can lead to delayed or missed checks, potentially resulting in prolonged exposure to moisture that may cause skin irritation, breakdown, or infection. The manual nature of this process also makes it challenging to maintain consistent care standards across different shifts and staff members.

Existing moisture detection systems for incontinence products have significant limitations. These systems typically employ reusable electronic devices that must be inserted into or attached to sanitary products and require battery-powered components that need regular charging or replacement, creating additional maintenance burdens for healthcare staff. The reusable nature of these devices raises sanitation concerns, as they must be cleaned and disinfected between uses. Furthermore, bulky electronic components may cause discomfort for wearers and interfere with the normal function of the sanitary product.

Healthcare facilities also struggle with inventory management of incontinence products. Manual tracking of product usage is time-consuming and prone to errors, often resulting in stockouts or excessive inventory levels. Without automated systems to monitor consumption patterns, facilities cannot accurately forecast supply needs or optimize their procurement processes.

Current waste management systems in healthcare environments lack the ability to verify proper disposal of soiled incontinence products. Traditional disposal methods provide no confirmation that products have been appropriately discarded, creating gaps in care documentation and compliance tracking. This limitation makes it difficult for facilities to maintain accurate records of care activities and demonstrate adherence to quality standards.

Family members of residents in long-term care facilities often express concerns about the quality of incontinence care their loved ones receive. The lack of transparent, real-time monitoring systems makes it challenging for families to have confidence in the care being provided. Additionally, healthcare administrators require better data on care delivery patterns to identify areas for improvement and ensure compliance with regulatory standards.

Incontinence care represents one of the most pervasive challenges in senior care, whether at home or in senior facilities. As an individual's health declines due to age or other factors, she or he may lose the ability to control their bladder, lose the mobility necessary to access a bathroom, or lose the mental capacity necessary to recognize their need to use the bathroom. Consequently, many seniors require incontinence products.

In senior facilities, residents typically wear sanitary products such as disposable briefs or diapers that are changed following urinary or bowel movements. Under the current standard of care model, staff checks resident wet/dry status at regular intervals. However, this process often leads to unfavorable circumstances. While the ideal process involves checking residents at regular intervals for urinary voids or bowel movements, changing them promptly, disposing of soiled sanitary products, and repeating the cycle, staff frequently fail to adhere strictly to this protocol. Such failures result in delayed or significantly delayed changes, leading to prolonged exposure to urine or feces on the resident's skin, which can ultimately cause skin breakdown and increase infection risk. Key issues with the current standard of care include staffing shortages and overburdened staff, which can cause oversight, negligence, or prioritization of other daily tasks over incontinence care, resulting in residents remaining wet for prolonged periods.

Current moisture monitoring systems have significant drawbacks. Existing brief monitoring systems require placement of a reusable “puck” or strip in the sanitary product, which is undesirable from a use perspective due to sanitation concerns. The need to recharge batteries is cumbersome and impractical in clinical environments. Furthermore, moisture sensors requiring battery-powered, attached devices may be uncomfortable to wear.

Families of residents in nursing home facilities would benefit from peace of mind knowing that their loved ones receive adequate care. Therefore, there is a need for a system to record and report quality of care metrics relevant to the resident.

Additional operational challenges exist in waste management and inventory control. Current waste disposal systems in healthcare facilities lack the ability to track and verify proper disposal of soiled incontinence products. Traditional waste bins provide no confirmation that soiled products have been properly disposed of, creating gaps in care documentation and compliance tracking. Healthcare facilities also struggle with inventory management of incontinence products, often experiencing stockouts or overstocking due to lack of automated usage tracking. Manual inventory counting is time-consuming, error-prone, and diverts staff attention from direct patient care. Without automated tracking of product usage and disposal, facilities cannot accurately forecast inventory needs or optimize supply chain management.

Therefore, there is a need for improved moisture detection systems that address these limitations. Specifically, there is a need for a moisture detection system that requires no traditional batteries or reusable electronics, allowing users to use sanitary products normally and dispose of them in a sanitary manner. Additionally, there is a need to provide real-time moisture monitoring alerts, including escalating alerts if not handled timely, and tracking facility performance in responding to those alerts. Furthermore, there is a need for intelligent disposal systems that can verify proper disposal of soiled products and automated inventory management systems that can track usage patterns and trigger reordering when supplies reach predetermined thresholds. Overall, there is a need for improved systems that can provide automated monitoring of moisture events while maintaining the simplicity and disposability that healthcare environments require.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a system for tracking hygiene products is provided. The system includes a plurality of hygiene products, each hygiene product having an associated tag. The system includes a reader configured to detect the tags associated with the hygiene products. The system includes a point of care unit configured to receive tracking information from the reader, wherein the point of care unit is further configured to track any of the time since a hygiene product was last replaced, moisture events, and rounding checks.

According to other aspects of the present disclosure, the system may include one or more of the following features. The tags may comprise radio frequency identification (RFID) tags. The reader may be configured to detect the RFID tags within a specified range. The point of care unit may comprise a display configured to show a dashboard interface indicating status information for multiple hygiene products. The status information may comprise at least one of: time since last change, moisture event detection, and rounding check completion. The system may further comprise a smart bin configured to detect disposal of soiled hygiene products. The smart bin may be further configured to update inventory information in response to detecting disposal of a soiled hygiene product.

According to another aspect of the present disclosure, a system for tracking hygiene products is provided. The system includes a plurality of hygiene products, each hygiene product having an associated tag. The system includes a reader configured to detect the tags associated with the hygiene products. The system includes a point of care unit configured to receive tracking information from the reader. The system includes an eBin configured to automatically track when new hygiene products are removed from a supply and when soiled hygiene products are disposed of, wherein the point of care unit is further configured to track any of the time since a hygiene product was last replaced, moisture events, and rounding checks.

According to other aspects of the present disclosure, the system may include one or more of the following features. The tags may comprise radio frequency identification (RFID) tags. The reader may be configured to detect the RFID tags within a specified range. The point of care unit may comprise a display configured to show a dashboard interface indicating status information for multiple hygiene products. The status information may comprise at least one of: time since last change, moisture event detection, and rounding check completion. The eBin may comprise a weight sensor configured to detect when a soiled hygiene product is disposed of based on a change in weight. The eBin may be further configured to update inventory information in response to detecting disposal of a soiled hygiene product.

According to another aspect of the present disclosure, a method for handling refuse or hygiene product disposal in a bin is provided. The method includes receiving a hygiene product in a bin. The method includes determining whether the hygiene product is an expired hygiene product. The method includes if the hygiene product is an expired hygiene product, detecting the expired hygiene product and displaying a message indicating an event recorded for a resident. The method includes if the hygiene product is not an expired hygiene product, determining whether the hygiene product is an active hygiene product. The method includes if the hygiene product is an active hygiene product, determining whether there are multiple hygiene products and prompting a user to select a resident.

According to other aspects of the present disclosure, the method may include one or more of the following features. The method may further include if the hygiene product is not an active hygiene product, increasing a net weight of the bin and sending a message payload indicating disposal of refuse. The method may further include after sending the message payload, prompting a user to select whether refuse was disposed of with the hygiene product. Determining whether the hygiene product is an expired hygiene product may comprise detecting whether the hygiene product transmits a signal. Detecting the expired hygiene product may comprise detecting a weight increase in the bin and determining that no signal is received from a hygiene product in the bin. The method may further include after detecting the expired hygiene product, providing an option for a user to indicate a false detection.

A system comprising a sanitary product, such as a diaper or brief, which detects the presence of moisture and sends a wireless signal indicating its moisture status is disclosed. In one embodiment, moisture may be determined upon activation of a circuit element. The circuit element triggers a signaling element which may transmit a signal to a detection device. The detection device, in some embodiments, logs the detection of moisture on a remote server. The system may provide an alert following the detection of moisture so that the sanitary product may be changed.

In one embodiment, the device incorporates small, disposable, battery-free sensors into sanitary products, such as incontinence briefs. When the sanitary product has absorbed a predetermined volume of fluid, the fluid may contact the sensor, activating and powering it to transmit a message that the sanitary product needs to be changed. Responsive to receiving the message, the system may provide the alert. It is contemplated that the alert may reduce the potential for adverse health effects from incontinence, improve the efficiency and job satisfaction of the care giver and preserve the dignity of the patient.

In accordance with one embodiment, the system comprises: a sensor integrated into a sanitary product associated with a wearer; a reader in communication with the sensor; and a point of care unit in communication with the reader, wherein the system is operative to carry out a method comprising the steps of: responsive to the sensor contacting moisture, the sensor transmits a signal; the reader receives the signal from the sensor and transmits the signal to the point of care unit; the point of care unit receives the signal from the reader and alerts a user of the detection of moisture in the sanitary product associated with the wearer; and responsive to the alert being cleared, wherein the alert cleared by the replacement of the sanitary product, associating a new sanitary product with the wearer, wherein the point of care unit tracks any of the time since the sanitary product was last replaced, moisture events, and rounding checks.

In one embodiment, the sensor is a motion detection sensor configured to be integrated into a sanitary product, comprising an integrated circuit comprising a first and second electrode, a capacitor, an RF transmitter, a power supply, and an adhesive to secure the sensor to the sanitary product; wherein upon moisture contacting the first and second electrodes the integrated circuit is completed, and the RF transmitter transmits a signal.

In an embodiment, the reader is configured as a transceiver to receive signals from moisture detection sensors and transmit the signals to a point of care unit, wherein the reader carries out a method comprising the steps of: identifying the presence of a moisture detection sensor associated with a wearer; detecting a signal transmitted from the moisture detection sensor, wherein the signal corresponds with a moisture event; responsive to the detecting the signal, transmitting a signal to a point of care unit of the wearer to alert a user of the moisture event; responsive to the moisture event being resolved, the reader does not receive the signal from the moisture detection sensor; and the reader identifying the presence of a new moisture detection sensor associated with the wearer.

In an aspect of the present disclosure, a system for monitoring incontinence care, comprising: a plurality of hygiene products or undergarments, each hygiene product or undergarment having an associated tag; a reader configured to detect the tags associated with the hygiene products or undergarments; a point of care unit configured to receive tracking information from the reader, wherein the point of care unit is further configured to track any of the time since a resident was last attended to, moisture events, and care events, wherein care events comprise checking the resident, changing the resident's hygiene product or undergarment, or toileting the resident, and wherein the point of care unit is configured to generate an alert when a resident has been unattended for longer than a predetermined time period.

In another aspect of the present disclosure, the system, wherein the tags comprise radio frequency identification (RFID) tags configured to be detected by the reader within a specified range.

In other aspects of the present disclosure, the system, wherein the point of care unit comprises a display configured to show a status icon indicating a wet status or a dry status for each resident.

In a further aspect of the present disclosure, the system, wherein the display of the point of care unit further indicates a room or bed for each resident.

In a further aspect of the present disclosure, the system, wherein the status icon further indicates an elapsed time since at least one of: a most recent moisture event detected for each of the plurality of hygiene products or undergarments, or a most recent care event performed for each resident associated with the plurality of hygiene products or undergarments.

In other aspects of the present disclosure, the system, further comprising a smart bin configured to detect disposal of soiled hygiene products or undergarments.

In a further aspect of the present disclosure, the system, wherein the smart bin is further configured to: detect a fluid volume of waste in the soiled hygiene products or undergarments; and determine, based on the fluid volume of waste, a hydration state of the resident.

In an aspect of the present disclosure, a sensor insert for a hygiene product, comprising: a non-woven material forming an uppermost layer; a super absorbent polymer layer positioned below the non-woven material; a tag positioned below the super absorbent polymer layer, the tag configured to detect moisture, wherein the super absorbent polymer layer is configured to consolidate the moisture at the tag; an inner adhesive layer positioned below the tag; and a back sheet positioned below the inner adhesive layer, wherein the sensor insert is configured to be adhered to an inside surface of the hygiene product.

In another aspect of the present disclosure, the sensor insert, further comprising an outer adhesive layer positioned below the back sheet.

In a further aspect of the present disclosure, the sensor insert, further comprising a release liner removably attached to the outer adhesive layer.

In other aspects of the present disclosure, the sensor insert, wherein the sensor insert further comprises an inner adhesive layer configured to secure the tag between the super absorbent polymer layer and the back sheet.

In other aspects of the present disclosure, the sensor insert, wherein the tag comprises a first electrode and a second electrode, wherein the tag is configured to detect moisture when the moisture bridges the first electrode and the second electrode.

In other aspects of the present disclosure, the sensor insert, wherein the sensor insert is configured to be positioned in a first location for a male patient and a second location for a female patient.

In a further aspect of the present disclosure, the sensor insert, wherein the first location is toward a front portion of the hygiene product and the second location is toward a middle portion of the hygiene product.

In an aspect of the present disclosure, a method for handling refuse or hygiene product disposal in a bin, comprising: receiving a hygiene product in a bin; determining the hygiene product is a soiled hygiene product; measuring a fluid volume of waste in the soiled hygiene product and displaying a message indicating an event recorded for a resident; determining, at least partially based on the fluid volume of waste, a hydration level of the resident.

In another aspect of the present disclosure, the method, further comprising tracking a fluid intake of the resident.

In a further aspect of the present disclosure, the method, wherein the determination of the hydration level of the resident is further based on the fluid intake of the resident.

In other aspects of the present disclosure, the method, wherein determining whether the hygiene product is the soiled hygiene product comprises detecting whether the hygiene product transmits a signal.

In a further aspect of the present disclosure, the method, wherein detecting the expired hygiene product comprises: detecting a weight increase in the bin; and determining that no signal is received from the hygiene product in the bin.

In a further aspect of the present disclosure, the method, further comprising: after detecting the soiled hygiene product, providing an option for a user to confirm the detection was accurately recorded.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a general eBrief system architecture, in accordance with one or more embodiments of the present disclosure.

FIG. 2 depicts a view of a moisture detection tag, in accordance with one or more embodiments of the present disclosure.

FIG. 3 illustrates a reader-assigned workflow with eTag, in accordance with one or more embodiments of the present disclosure.

FIG. 4A depicts an eBrief assembly process, in accordance with one or more embodiments of the present disclosure.

FIG. 4B depicts an eBrief assembly process, in accordance with one or more embodiments of the present disclosure.

FIG. 5A illustrates an alternative eBrief assembly process, in accordance with one or more embodiments of the present disclosure.

FIG. 5B illustrates an alternative eBrief assembly process, in accordance with one or more embodiments of the present disclosure.

FIG. 6A depicts another alternative eBrief assembly process, in accordance with one or more embodiments of the present disclosure.

FIG. 6B depicts another alternative eBrief assembly process, in accordance with one or more embodiments of the present disclosure.

FIG. 7 illustrates a cross-sectional view of an eBrief showing potential tag placement locations, in accordance with one or more embodiments of the present disclosure.

FIG. 8A depicts a detailed cross-sectional view of an eBrief layer composition, in accordance with one or more embodiments of the present disclosure.

FIG. 8B depicts a detailed cross-sectional view of an eBrief layer composition, in accordance with one or more embodiments of the present disclosure.

FIG. 8C depicts a detailed cross-sectional view of an eBrief layer composition, in accordance with one or more embodiments of the present disclosure.

FIG. 9 illustrates a cross-sectional view of a layered absorbent product structure, in accordance with one or more embodiments of the present disclosure.

FIG. 10 illustrates a cross-sectional view of a layered absorbent product structure, in accordance with one or more embodiments of the present disclosure.

FIG. 11A depicts an alternative cross-sectional view of a layered absorbent product structure, in accordance with one or more embodiments of the present disclosure.

FIG. 11B depicts an alternative cross-sectional view of a layered absorbent product structure, in accordance with one or more embodiments of the present disclosure.

FIG. 12 illustrates a tag encased in a water-soluble film inside an eBrief, in accordance with one or more embodiments of the present disclosure.

FIG. 13 depicts a tag having a capacitor, in accordance with one or more embodiments of the present disclosure.

FIG. 14 illustrates an exploded view of a moisture detection system with a lateral flow membrane, in accordance with one or more embodiments of the present disclosure.

FIG. 15 depicts a tag with electrode spacing, in accordance with one or more embodiments of the present disclosure.

FIG. 16 illustrates an eBrief with multiple distributed tags, in accordance with one or more embodiments of the present disclosure.

FIG. 17 depicts a cross-sectional view of a sanitary product layer architecture, in accordance with one or more embodiments of the present disclosure.

FIG. 18 depicts a cross-sectional view of a sanitary product layer architecture, in accordance with one or more embodiments of the present disclosure.

FIG. 19A illustrates an alternative layering configuration for integrating tags into sanitary products, in accordance with one or more embodiments of the present disclosure.

FIG. 19B illustrates an alternative layering configuration for integrating tags into sanitary products, in accordance with one or more embodiments of the present disclosure.

FIG. 19C illustrates an alternative layering configuration for integrating tags into sanitary products, in accordance with one or more embodiments of the present disclosure.

FIG. 19D illustrates an alternative layering configuration for integrating tags into sanitary products, in accordance with one or more embodiments of the present disclosure.

FIG. 20A depicts a reader disguised as artwork, in accordance with one or more embodiments of the present disclosure.

FIG. 20B depicts a reader disguised as artwork, in accordance with one or more embodiments of the present disclosure.

FIG. 20C depicts a reader disguised as artwork, in accordance with one or more embodiments of the present disclosure.

FIG. 21 illustrates an orthogonal view of a wearable reader, in accordance with one or more embodiments of the present disclosure.

FIG. 22 depicts an alternative eBrief system architecture, in accordance with one or more embodiments of the present disclosure.

FIG. 23 illustrates a system diagram showing signals from multiple moisture monitoring systems, in accordance with one or more embodiments of the present disclosure.

FIG. 24 depicts a reader-assigned workflow, in accordance with one or more embodiments of the present disclosure.

FIG. 25 illustrates a flowchart for a tag-ID assigned workflow, in accordance with one or more embodiments of the present disclosure.

FIG. 26 depicts a scanner-assigned workflow, in accordance with one or more embodiments of the present disclosure.

FIG. 27 illustrates an auto-assigned workflow, in accordance with one or more embodiments of the present disclosure.

FIG. 28 depicts a dataflow and connectivity architecture, in accordance with one or more embodiments of the present disclosure.

FIG. 29 illustrates a network architecture for data communication, in accordance with one or more embodiments of the present disclosure.

FIG. 30 depicts a moisture detection system with multiple tags, in accordance with one or more embodiments of the present disclosure.

FIG. 31 illustrates a detailed engineering drawing view of a tag, in accordance with one or more embodiments of the present disclosure.

FIG. 32 depicts eBrief tag assembly processes, in accordance with one or more embodiments of the present disclosure.

FIG. 33 illustrates a flowchart for handling refuse or eBrief disposal in a bin, in accordance with one or more embodiments of the present disclosure.

FIG. 34 depicts a system architecture for a smart bin monitoring system, in accordance with one or more embodiments of the present disclosure.

FIG. 35 illustrates a flowchart for discarding soiled eBriefs, in accordance with one or more embodiments of the present disclosure.

FIG. 36A depicts an exploded view of a sensor insert, in accordance with one or more embodiments of the present disclosure.

FIG. 36B depicts an exploded view of a sensor insert, in accordance with one or more embodiments of the present disclosure.

FIG. 37 illustrates a method for applying a sensor insert, in accordance with one or more embodiments of the present disclosure.

FIG. 38A depicts a reader, in accordance with one or more embodiments of the present disclosure.

FIG. 38B depicts an exploded view of a reader, in accordance with one or more embodiments of the present disclosure.

FIG. 39 illustrates a flowchart for handling refuse or hygiene product disposal in a bin, in accordance with one or more embodiments of the present disclosure.

FIG. 40 depicts a flowchart for handling multiple hygiene product disposals and user corrections, in accordance with one or more embodiments of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

These and other aspects, features, and advantages of the present invention will become more readily apparent from the following drawings and the detailed description of the preferred embodiments.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

The present disclosure is directed to a system and apparatus for moisture event detection. More particularly, to an electronic incontinence monitoring system, apparatus, and associated methods. In one embodiment, the system may comprise moisture sensors having an electronic transmission capability and external means for receiving that transmission to sense the presence of moisture, for example, moisture from bodily fluid voiding events.

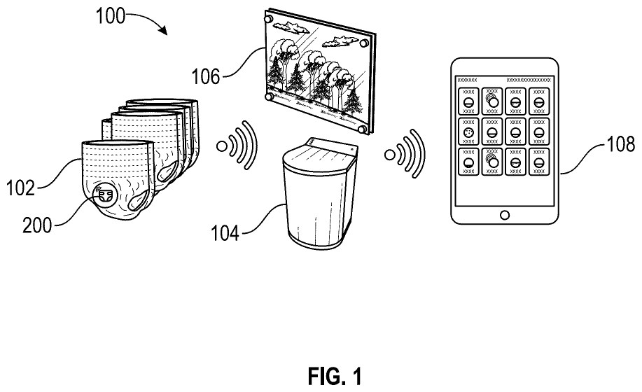

The system may comprise a sanitary product having an electronic sensor, a reader, and a point of care unit. One example of the system architecture is shown in FIG. 1 herein.

As used herein, the term “eBrief” may refer to a hygiene product, such as a diaper, brief, or underwear, that is configured to have a tag inserted into or integrated with it. The tag may be capable of detecting moisture and transmitting signals.

As used herein, the term “eBin” may refer to a waste receptacle or trash container that is equipped with capabilities to monitor and track items placed inside of it or removed from it. The eBin may include sensors, weight measurement systems, or other mechanisms to identify and record the disposal or removal of objects such as used or new hygiene products.

The present disclosure relates to a moisture detection system for use in healthcare applications. The system may include a moisture sensor integrated into a sanitary product, a reader device, and a point of care unit. The moisture sensor may be configured to detect the presence of moisture in the sanitary product and transmit a signal upon detection. The reader device may be configured to receive the signal from the moisture sensor and relay information to the point of care unit. The point of care unit may provide alerts and tracking capabilities related to moisture events.

In some cases, the system may be used for incontinence care monitoring in healthcare facilities or home care settings. The moisture detection system may allow for real-time alerts when moisture is detected in a sanitary product, enabling timely changes and improved patient care. The system may also provide data logging and analysis capabilities to track moisture events over time.

The moisture sensor may utilize various detection mechanisms and may be designed for single-use integration into disposable sanitary products. The reader device may take different form factors to suit various environments. The point of care unit may include software for managing alerts, tracking data, and generating reports related to moisture events and care activities.

FIG. 1 illustrates an eBrief system architecture 100 in accordance with one or more embodiments of the present disclosure. The eBrief system architecture 100 may comprise an eBrief 102, an eBin 104, a reader 106, and a point of care unit 108.

The eBrief 102 may be a sanitary product, such as a diaper or brief, which incorporates a tag 200. In some cases, the tag 200 may be configured to detect the presence of moisture and send a wireless signal indicating the moisture status of the eBrief 102.

The reader 106 may be in communication with the eBrief 102. In some cases, the reader 106 may be configured to receive signals from the tag 200 when moisture is detected in the eBrief 102. The reader 106 may then relay these signals to other components of the eBrief system architecture 100.

The eBin 104 may also be in communication with the eBrief 102. In some cases, the eBin 104 may be used for disposal of soiled eBriefs 102. The eBin 104 may be configured to detect when a soiled eBrief 102 has been disposed of and communicate this information to other components of the eBrief system architecture 100.

The point of care unit 108 may be in communication with both the reader 106 and the eBin 104. In some cases, the point of care unit 108 may be configured to receive and process information from the reader 106 and the eBin 104. The point of care unit 108 may use this information to alert caregivers when an eBrief 102 needs to be changed.

In operation, when moisture is detected in the eBrief 102, the tag 200 may send a signal to the reader 106. The reader 106 may then relay this information to the point of care unit 108. The point of care unit 108 may alert a caregiver that the eBrief 102 needs to be changed. After changing the eBrief 102, the soiled eBrief 102 may be disposed of in the eBin 104. The eBin 104 may detect this disposal and communicate this information to the point of care unit 108, which may then update its records.

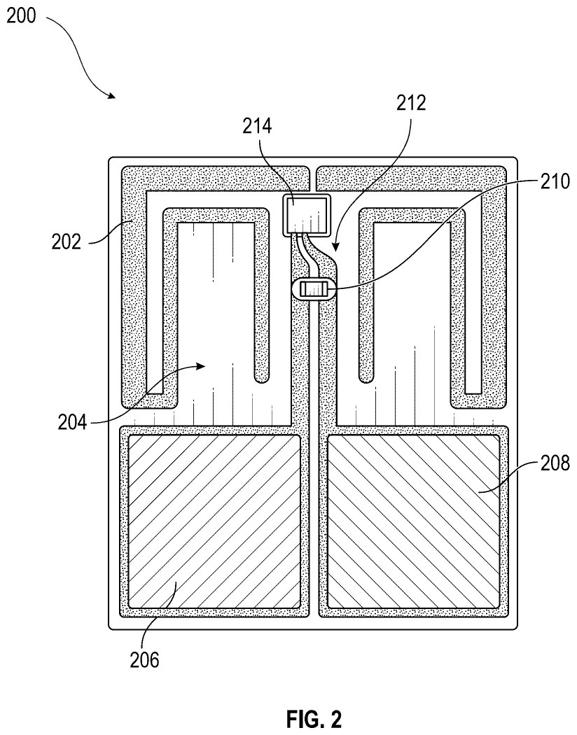

FIG. 2 illustrates a view of a moisture detection tag 200. The tag 200 comprises an antenna 202 that extends across a portion of the tag's surface. A topcoat dielectric 204 may be disposed over portions of the tag 200. The tag 200 includes a silver electrode 206 and a magnesium electrode 208 positioned on the surface. A capacitor 210 may be mounted on the tag 200 and connected to other components. An epoxy dielectric coating 212 may cover portions of the tag 200. The tag 200 also includes an integrated circuit 214 mounted on its surface and electrically connected to other components.

In some cases, the antenna 202 may be configured to transmit signals when the tag 200 is activated by moisture. The topcoat dielectric 204 may provide protection and insulation for the underlying components. The silver electrode 206 and magnesium electrode 208 may form a galvanic cell when in contact with moisture, generating a small electrical current.

The capacitor 210 may store electrical charge and help regulate the power supply to other components. The epoxy dielectric coating 212 may provide additional protection and insulation for sensitive components. The integrated circuit 214 may control the overall function of the tag 200, processing signals and managing power distribution.

In some cases, when moisture bridges the silver electrode 206 and magnesium electrode 208, a small electrical current may be generated. This current may be used to power the integrated circuit 214. The integrated circuit 214 may then activate the antenna 202 to transmit a signal indicating the presence of moisture.

The components of the tag 200 may be arranged in layers to form a thin, flexible structure suitable for integration into sanitary products. The layered construction may allow for efficient use of space while providing the necessary functionality for moisture detection and signal transmission.

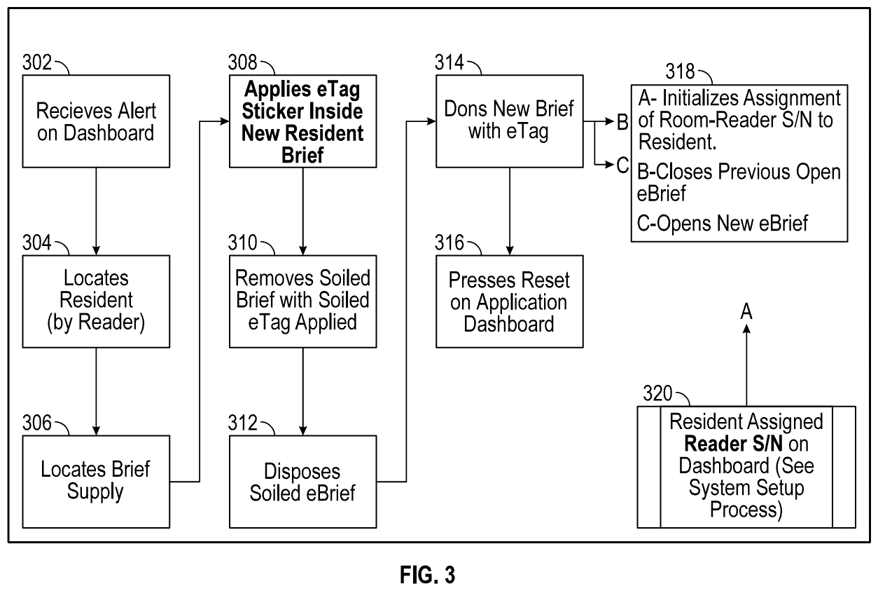

FIG. 3 illustrates a reader-assigned workflow with eTag 300. The reader-assigned workflow with eTag 300 may comprise a series of steps for managing moisture detection and sanitary product changes.

The reader-assigned workflow with eTag 300 may begin with a step 302, where a dashboard receives a fluid detection alert. This alert may indicate that moisture has been detected in a sanitary product worn by a resident.

Following the alert, a step 304 may involve a reader identifying and locating the resident with the tag giving the fluid detection signal. The reader may use the signal from the activated tag to determine which resident requires attention.

The reader-assigned workflow with eTag 300 may then proceed to a step 306, where a supply of clean eBriefs is located. This step may ensure that a caretaker has the necessary materials for changing the resident's sanitary product.

A step 308 may involve orienting and applying a brief tag into the eBrief. This step may prepare the new sanitary product for use and enable it to be tracked by the system.

The reader-assigned workflow with eTag 300 may continue with a step 310, where the soiled eBrief is removed from the resident. This step may be performed by a caretaker to prepare for applying a clean sanitary product.

A step 312 may involve disposing of the soiled eBrief. The caretaker may place the used sanitary product in an appropriate disposal container.

The reader-assigned workflow with eTag 300 may then move to a step 314, where the resident dons the new eBrief and tag. This step may involve the caretaker assisting the resident in putting on the clean sanitary product.

A step 316 may include resetting the application dashboard for the particular resident and starting to track the new tag. This step may update the system to monitor the newly applied sanitary product.

The reader-assigned workflow with eTag 300 may include a step 318 with multiple sub-steps. In these sub-steps, the reader may initialize an assignment of a new identification number to the resident's new tag, terminate the previous tag, and open a new eBrief to be tracked.

A final step 320 may involve the application dashboard resetting the system relative to the particular resident and starting to track the tag. This step may ensure that the system is fully updated and ready to monitor the new sanitary product.

In some cases, the reader-assigned workflow with eTag 300 may enable efficient management of moisture detection and sanitary product changes, allowing caretakers to respond promptly to residents'needs while maintaining accurate tracking of sanitary products in use.

The eBrief may be created by integrating a tag into existing sanitary products through various assembly processes. Two alternative eBrief assembly processes are described herein.

In a first process, referred to as an eBrief assembly process A 400, a backsheet of a hygiene product may be cut in a step 402. The process 400 may then proceed to a step 404, where an original tag may be inserted into the hygiene product. Following this, a step 406 may involve orienting and applying a brief tag into the hygiene product. The eBrief assembly process A 400 may conclude with a step 408, where a finished eBrief may be produced.

An alternative process, referred to as an eBrief assembly process B 500, may begin with a step 502, where a window may be cut in a backsheet of a hygiene product. The process 500 may then move to a step 504, where an original tag may be applied onto the backsheet. The eBrief assembly process B 500 may conclude with a step 506, where a finished eBrief may be produced.

The two processes differ primarily in their initial approach to modifying the hygiene product and the subsequent placement of the tag. In the eBrief assembly process A 400, the backsheet may be cut to create an opening for inserting the tag, while in the eBrief assembly process B 500, a window may be cut in the backsheet to which the tag may be applied directly. Both processes may result in the creation of an eBrief, integrating a tag into an existing sanitary product.

The present disclosure provides a third alternative method for assembling an eBrief, referred to as eBrief assembly process C 600. This process differs from the previously described methods by folding the hygiene product around the tag instead of cutting the hygiene product open.

eBrief assembly process C 600 begins with a step 602, where a hygiene product may be opened and unfolded. This initial step prepares the hygiene product for tag insertion without requiring any cutting or modification of the product itself.

Following the unfolding, a step 604 involves applying a tag 200 to an inside surface of the hygiene product. In some cases, the tag 200 may be positioned in a location that optimizes moisture detection while minimizing discomfort to the wearer. The placement of the tag 200 directly on the inside surface may allow for more immediate moisture detection compared to methods where the tag 200 is inserted into or between layers of the hygiene product.

After tag 200 application, a step 606 involves refolding the hygiene product. This step may ensure that the tag 200 remains securely in place and that the hygiene product returns to its original form factor. The refolding process may be performed carefully to avoid disturbing the positioning of the tag 200.

The eBrief assembly process C 600 concludes with a step 608, where a finished eBrief may be produced. The resulting eBrief incorporates the moisture-sensing capabilities of the tag 200 while maintaining the original structure and comfort of the hygiene product.

This method may offer several advantages over the previously described assembly processes. By avoiding the need to cut or permanently alter the hygiene product, eBrief assembly process C 600 may preserve the integrity of the product's moisture barriers and reduce the risk of leakage. Additionally, this process may be more suitable for retrofitting existing hygiene products with moisture-sensing capabilities, potentially allowing for greater flexibility in product selection and inventory management.

The eBrief may comprise multiple layers arranged in a stacked configuration. A cross-sectional view 800 of an eBrief cross-section 702 may show these various layers. From top to bottom, these layers may include a top sheet 706, an acquisition distribution layer 708, an absorbent core 712, and a back sheet 716.

In some cases, the eBrief cross-section 702 may include potential tag 200 placement locations. A first tag location 704 may be positioned near the top sheet 706. A second tag location 710 may be positioned between the acquisition distribution layer 708 and the absorbent core 712. A third tag location 714 may be positioned between the absorbent core 712 and the back sheet 716.

The cross-sectional view 800 may show a detailed layer composition of the eBrief. The layers may include a non-woven material 802, a positive electrode 804a and a negative electrode 804b of a tag 200, and an adhesive 808. The structure may further comprise an acquisition layer 810 and a distribution layer 812. Below these layers may be an inner core wrap 814 containing a super absorbent polymer 816, followed by an outer core wrap 818 and a back sheet 820.

In some cases, the tag 200 may be integrated into the eBrief at different positions within the layer structure. This integration may allow for different moisture detection configurations based on the placement location. For example, placing the tag 200 at the first tag location 704 near the top sheet 706 may allow for quicker moisture detection, as the tag 200 may be closer to the source of moisture. Placing the tag 200 at the second tag location 710 or the third tag location 714 may allow for detection of higher levels of moisture saturation in the eBrief.

The tag 200 may be oriented in different ways within the eBrief. In some cases, the tag 200 may be placed “face-up” with the positive electrode 804a and negative electrode 804b facing towards the top sheet 706. In other cases, the tag 200 may be placed “face-down” with the electrodes facing towards the back sheet 820. These different orientations may affect how quickly or at what level of saturation the tag 200 detects moisture.

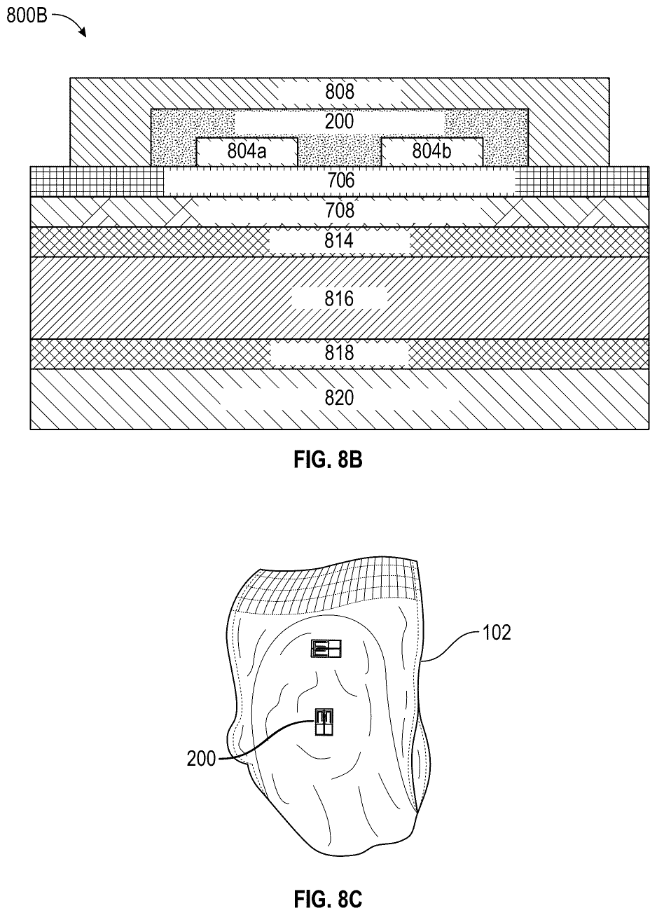

FIGS. 8B-8C illustrate alternative layer compositions of an eBrief 102. FIG. 8B depicts a detailed cross-sectional view 800 showing one alternative layer composition of the eBrief 102. The layers include an adhesive layer 808 securing a positive electrode 804a and a negative electrode 804b of a tag 200 to an acquisition layer 810. The structure further comprises a distribution layer 812, an inner core wrap 814 containing a super absorbent polymer 816, followed by an outer core wrap 818 and a back sheet 820.

In some cases, the arrangement of layers in FIG. 8B may be referred to as eBrief layer composition A 800A. This composition places the tag 200 near the top of the eBrief 102, which may allow for quicker detection of moisture as it enters the eBrief 102.

FIG. 8C illustrates another alternative layer composition, which may be referred to as eBrief layer composition B 800B. In this configuration, the tag 200 may be positioned lower in the layer structure of the eBrief 102. The specific arrangement of layers in eBrief layer composition B 800B may vary, but may include similar components to those shown in eBrief layer composition A 800A.

The cross-sectional views 800 illustrate how the tag 200 can be integrated at different positions within the layer structure of the eBrief 102, allowing for different moisture detection configurations based on the placement location. In some cases, placing the tag 200 closer to the top layers of the eBrief 102, as shown in eBrief layer composition A 800A, may result in faster moisture detection. Conversely, positioning the tag 200 lower in the layer structure, as may be the case in eBrief layer composition B 800B, may provide detection of more significant moisture events.

The specific arrangement of layers and positioning of the tag 200 may affect the performance of the moisture detection system. For example, the placement of the tag 200 relative to the super absorbent polymer 816 and other absorbent layers may influence how quickly and at what level of saturation the tag 200 detects moisture. In some cases, these different configurations may allow for customization of the eBrief 102 to suit various needs or preferences in moisture detection sensitivity and timing.

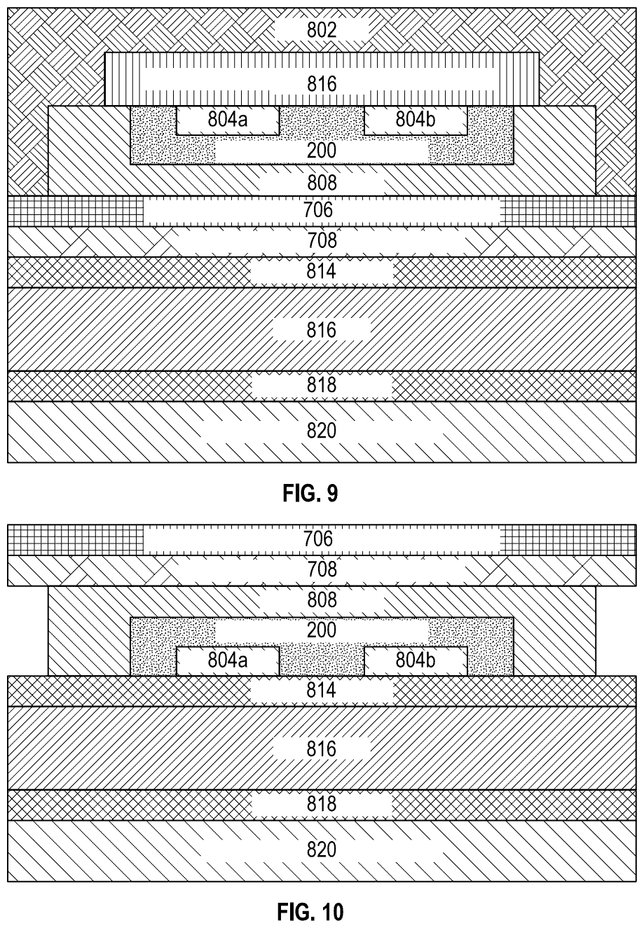

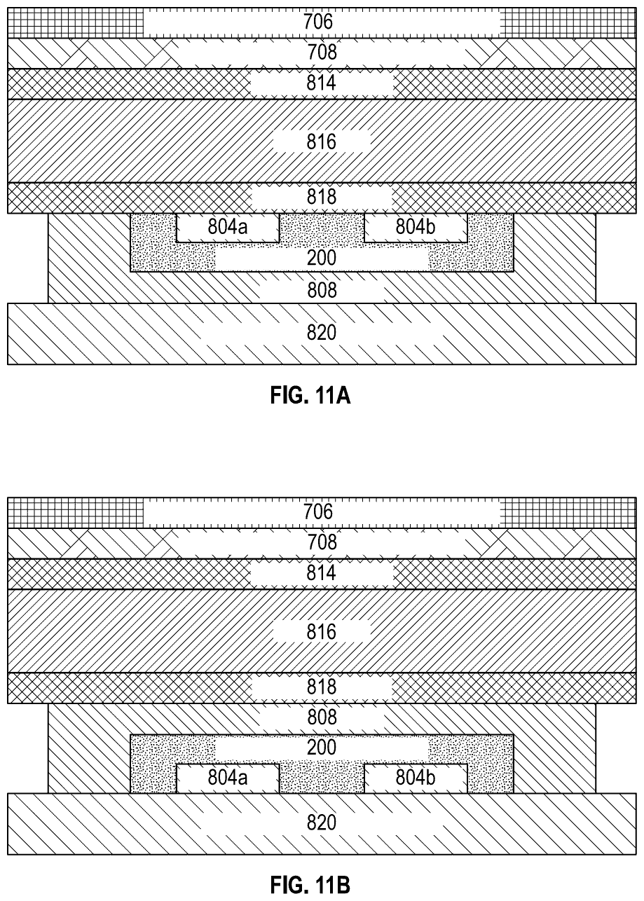

FIG. 9 and FIG. 10 illustrate cross-sectional views of layered absorbent product structures incorporating a tag 200 for moisture detection.

In FIG. 9, the structure includes a non-woven material 802 at the uppermost layer. Below the non-woven material 802 is the tag 200 including a positive electrode 804a and a negative electrode 804b positioned adjacent to each other. An adhesive layer 808 secures these components together.

The structure continues with an acquisition layer 810 and a distribution layer 812. Below these layers is an inner core wrap 814 that contains a super absorbent polymer 816. The super absorbent polymer 816 is further enclosed by an outer core wrap 818. A back sheet 820 forms the outermost layer of the structure.

In this configuration, the tag 200 is integrated into the layered structure between the non-woven material 802 and the adhesive layer 808, with the positive electrode 804a and negative electrode 804b forming part of the tag's sensing mechanism. The layers may be arranged to facilitate fluid movement from the non-woven material 802 through the various layers while maintaining structural integrity through the adhesive 808.

FIG. 10 illustrates another cross-sectional view of a layered absorbent product structure. In this configuration, the structure includes an acquisition layer 810 and a distribution layer 812 at the uppermost layers. The structure continues with an adhesive layer 808 adhering the tag 200 to the inner core wrap 814.

Similar to FIG. 9, the structure in FIG. 10 includes an inner core wrap 814 that contains a super absorbent polymer 816. The super absorbent polymer 816 is further enclosed by an outer core wrap 818. A back sheet 820 forms the outermost layer of the structure.

In some cases, the tag 200 may include an absorbent layer to increase moisture retention time. This absorbent layer may be positioned adjacent to the electrodes of the tag 200, allowing for prolonged contact with moisture and potentially enhancing the sensitivity of the moisture detection system.

The layered structures shown in FIG. 9 and FIG. 10 may provide different moisture detection capabilities based on the positioning of the tag 200 within the layers. The configuration in FIG. 9, with the tag 200 closer to the non-woven material 802, may allow for quicker detection of moisture. The configuration in FIG. 10, with the tag 200 positioned deeper within the structure, may provide detection of more significant moisture events.

FIGS. 11A-11B illustrate alternative embodiments of cross-sectional views of a layered absorbent product structure. The structures include an acquisition layer 810 and a distribution layer 812. Below these layers is an inner core wrap 814 that contains a super absorbent polymer 816. The super absorbent polymer 816 is further enclosed by an outer core wrap 818.

In FIG. 11A, the structure continues with a tag 200 being adhered to the outer core wrap 818 by an adhesive 808. A back sheet 820 forms the outermost layer of the structure. The tag 200 includes a positive electrode 804a and a negative electrode 804b.

In FIG. 11B, the structure continues with the adhesive 808 adhering the tag 200 to the back sheet 820 which forms the outermost layer of the structure. Similar to FIG. 11A, the tag 200 includes a positive electrode 804a and a negative electrode 804b.

The positioning of the tag 200 in these configurations may affect moisture detection sensitivity and accuracy. In FIG. 11A, the tag 200 is positioned between the outer core wrap 818 and the back sheet 820. This configuration may allow the tag 200 to detect moisture after it has passed through the inner layers of the absorbent product. In some cases, this placement may result in detection of higher levels of moisture saturation.

In FIG. 11B, the tag 200 is positioned directly on the back sheet 820. This configuration may allow for earlier detection of moisture as it reaches the outermost layer of the absorbent product. In some cases, this placement may provide faster response times for moisture detection.

The layered structures in both configurations may work together to manage fluid absorption and distribution. The acquisition layer 810 and distribution layer 812 may help to quickly spread fluid across the absorbent product. The super absorbent polymer 816 contained within the inner core wrap 814 and outer core wrap 818 may absorb and retain large amounts of fluid.

In some cases, the positioning of the tag 200 in relation to these absorbent layers may be selected based on desired moisture detection characteristics. The configuration in FIG. 11A may be suitable for detecting higher levels of saturation, while the configuration in FIG. 11B may be more sensitive to initial moisture presence.

The eBrief may incorporate various methods for controlling moisture detection sensitivity. In some cases, a water soluble film may be disposed over electrodes of a tag to create an activation threshold. The water soluble film may be configured to dissolve after exposure to sufficient fluid and/or time, permitting access to the electrodes. A tag comprising a water soluble film may reduce the probability of a small or insignificant void activating the sensor. Instead, sufficient fluid volume may be required to dissolve the water soluble film, exposing the electrodes, and activating the tag.

In some embodiments, a tag may comprise a capacitor to increase the liquid exposure time and/or volume required to activate the tag. The capacitor may have different levels of capacitance. In some cases, the capacitor may have a higher capacitance requiring more time to activate. In other cases, the capacitor may have a lower capacitance requiring less time to activate. The capacitor may need to charge to a sufficient voltage prior to achieving activation voltage of the tag. This charging effect may delay the onset of the signal by taking additional time to increase circuit voltage to the necessary voltage. If circuit voltage is not increased to a minimum value fast enough, then the absorbency of the hygiene product may draw fluid away from the sensor before activation. The tag may be activated once sufficient fluid is in contact with the sensor and the capacitor charging time can be achieved.

An eBrief may incorporate a lateral flow membrane to delay fluid transport to electrodes of a tag. The lateral flow membrane may kinetically limit fluid transport to the tag surface. The porosity and/or hydrophobicity of the membrane may be adjusted such that a quantity of moisture greater than a threshold is required to activate the sensor. In some embodiments, the lateral flow membrane may be oriented in a pattern which extends to various parts of the eBrief to monitor other endpoints. For example, the lateral flow membrane may extend outward in a finger-like pattern, with multiple fingers radiating from a central area where the tag is positioned. The tag may be situated between an inner eBrief layer and an outer eBrief layer. The lateral flow membrane may be configured in a pattern that allows fluid to travel along the finger portions toward the tag. Such an arrangement may allow early detection of moisture by concentrating moisture spread throughout an area to the tag.

Each of these approaches—the water soluble film, capacitor, and lateral flow membrane—offers different benefits for controlling moisture detection sensitivity. The water soluble film approach may provide a simple physical barrier that dissolves with sufficient moisture. The capacitor method may allow for electronic tuning of the activation delay. The lateral flow membrane may offer spatial control over moisture detection across the eBrief.

In some cases, an eBrief may include a tag with an electrode spacing between a first electrode and a second electrode. The electrode spacing may affect the sensitivity of moisture detection in the eBrief. A larger electrode spacing may require a greater area of saturation in the eBrief to complete an electrical connection between the first electrode and the second electrode. This configuration may reduce the likelihood of false activations from small amounts of moisture.

In some implementations, the electrode spacing may be adjusted to tune the moisture detection sensitivity. A smaller electrode spacing may allow for detection of smaller amounts of moisture, while a larger electrode spacing may require more substantial saturation before triggering a moisture detection event.

In some cases, an eBrief may incorporate multiple tags distributed throughout the eBrief. The use of multiple tags may provide several potential benefits. For example, multiple tags may allow for detection of moisture in different areas of the eBrief. This configuration may enable more precise localization of moisture within the eBrief.

Additionally, the use of multiple tags may increase the reliability of moisture detection. In some implementations, the eBrief system may require activation of a certain number or percentage of tags before signaling a moisture event. This approach may further reduce the likelihood of false positives in moisture detection.

The distribution of multiple tags throughout an eBrief may also allow for differentiation between different types of moisture events. For example, the pattern of tag activations may potentially distinguish between urinary voids and bowel movements based on the location and extent of moisture detected.

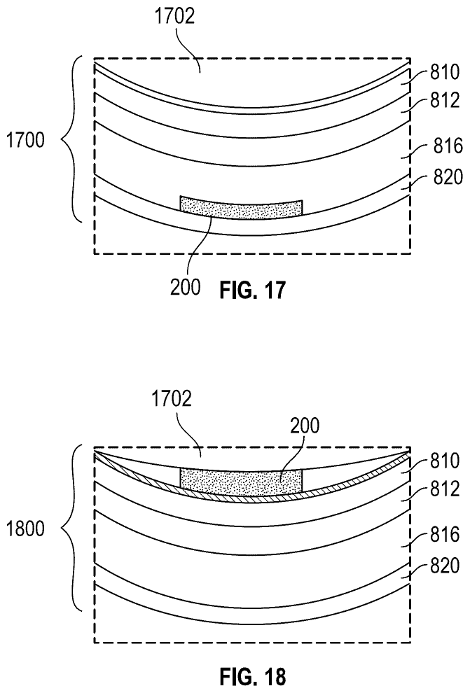

In some cases, an eBrief may comprise multiple layers arranged in different configurations to optimize moisture detection and user comfort. FIG. 17 and FIG. 18 illustrate cross-sectional views of two example layer architectures for an eBrief.

FIG. 17 depicts eBrief layers 1700 arranged in a first configuration. The eBrief layers 1700 may include a skin 1702 at the topmost layer, which may be in direct contact with a wearer's body. Below the skin 1702, an acquisition layer 810 and a distribution layer 812 may be positioned to quickly absorb and spread moisture. A super-absorbent layer 922 may be located beneath the distribution layer 812 to retain fluid. A back sheet 820 may form the outermost layer of the eBrief layers 1700. In this configuration, a tag 200 may be secured between the super-absorbent layer 922 and the back sheet 820.

FIG. 18 shows eBrief layers 1800 arranged in a second configuration. Similar to the first configuration, the eBrief layers 1800 may include a skin 1702 at the top, followed by an acquisition layer 810, a distribution layer 812, a super-absorbent layer 922, and a back sheet 820. However, in this arrangement, the tag 200 may be positioned between the distribution layer 812 and the skin 1702.

The placement of the tag 200 in each configuration may affect moisture detection performance. In the configuration of FIG. 17, the tag 200 may detect moisture after it has been absorbed by the super-absorbent layer 922, potentially providing a measure of saturation level. In the configuration of FIG. 18, the tag 200 may detect moisture more quickly due to its proximity to the skin 1702.

In some cases, placing the tag 200 closer to the skin 1702 may allow for faster moisture detection but may impact user comfort. Conversely, positioning the tag 200 further from the skin 1702 may enhance comfort while potentially delaying moisture detection.

The eBrief layers 1700 and 1800 may work together to manage fluid absorption and distribution while enabling moisture detection through the strategically placed tag 200. The specific arrangement of layers and tag placement may be selected based on desired performance characteristics and user comfort considerations.

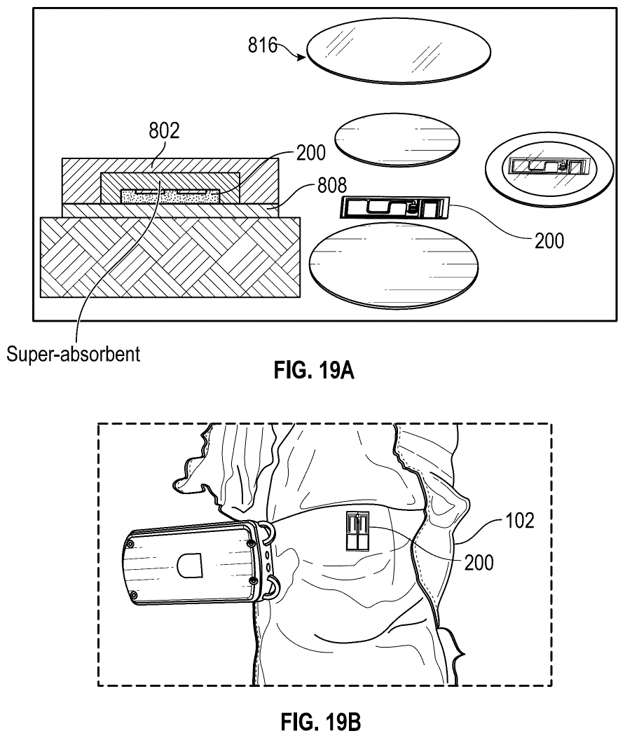

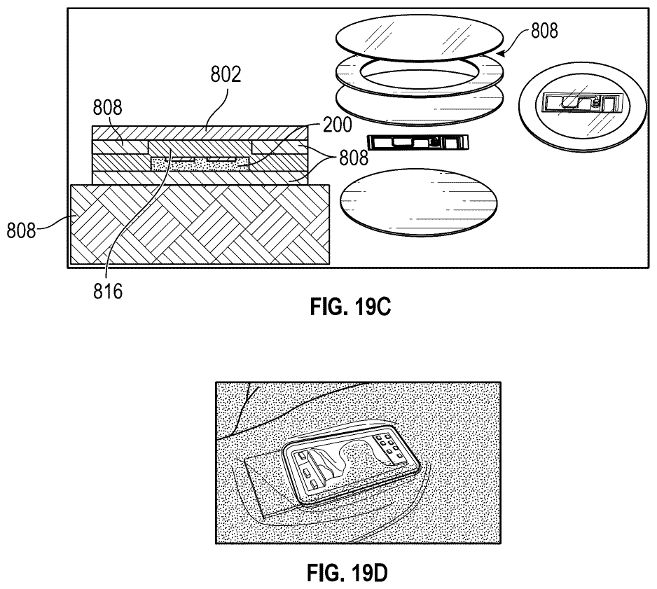

FIGS. 19A, 19B, 19C, and 19D illustrate alternative layering structures for a moisture detection system. These configurations may enhance moisture detection capabilities and overall product performance.

In FIGS. 19A and 19B, an alternative layering structure is shown. The structure begins with a non-woven material 802, followed by a super-absorbent layer 816. A tag 200 is positioned on top of the super-absorbent layer 816. In some cases, a silver electrode 206 may be incorporated into the tag 200 to facilitate moisture detection. An adhesive may be used to secure the tag 200 and layers to an eBrief 102.

FIGS. 19C and 19D depict a cross-sectional view of another embodiment of a layered structure showing the integration of a tag 200 into a sanitary product. The tag 200 is positioned between multiple layers. In some cases, the tag 200 may be placed adjacent to a non-woven hydrophilic layer 922/816. An adhesive layer 904 may secure the components together. The layered structure may be integrated into a diaper 1022.

The arrangement of layers in these configurations may allow for effective moisture detection while maintaining the comfort and functionality of the sanitary product. The positioning of the tag 200 between absorbent layers may enable accurate sensing of moisture levels. The use of adhesives may ensure the stability of the layered structure during use.

In some cases, the super-absorbent layer 922 may enhance the moisture retention capabilities of the product. The non-woven hydrophilic layer 922/816 may facilitate the movement of moisture towards the tag 200, potentially improving detection speed and accuracy.

These alternative layering structures demonstrate various approaches to integrating moisture detection technology into sanitary products. The specific arrangement of layers and components may be selected based on factors such as desired sensitivity, product type, and manufacturing considerations.

The system may include a reader 106 configured in various form factors to accommodate different care environments and usage scenarios. In some cases, a reader 106 may be wall-mounted and concealed as artwork 2006. The reader 106 may be housed within a housing 2002 that can be disguised as a decorative item or integrated into existing room decor. For example, the reader 106 may be hidden behind a painting or photograph mounted on a wall. This configuration allows the reader 106 to blend seamlessly into the care environment while maintaining its functionality.

In other cases, the system may utilize a wearable reader 2106. The wearable reader 2106 may be designed as a portable device that can be worn on the body, such as on the wrist. The wearable reader 2106 may provide the same functionality as the wall-mounted reader 106 but in a compact, mobile form factor. This configuration may allow caregivers to maintain connectivity with the moisture detection system even when moving between different areas of a care facility or when providing care outside of fixed reader locations.

The wall-mounted reader 106 concealed as artwork 2006 may be particularly suitable for residential care settings or facilities where maintaining a home-like atmosphere is desired. The discreet nature of this configuration may help preserve the dignity of care recipients by minimizing the visible presence of medical monitoring equipment.

The wearable reader 2106, on the other hand, may be especially useful in dynamic care environments where caregivers are frequently moving between different areas or providing care to multiple individuals. The portability of the wearable reader 2106 may enable continuous monitoring capabilities regardless of the caregiver's location within a facility.

Both reader configurations may be designed to communicate with the moisture detection sensors and relay information to the point of care unit. The choice between a wall-mounted reader 106 or a wearable reader 2106 may depend on factors such as the specific care setting, mobility requirements of caregivers, and preferences for integrating technology into the care environment.

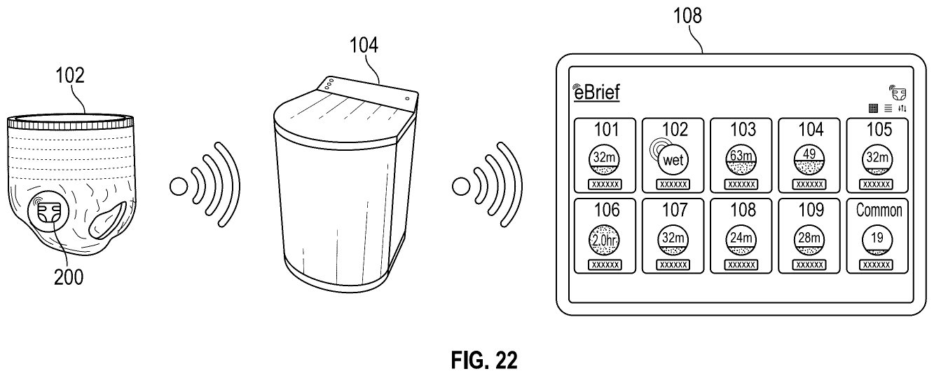

FIG. 22 illustrates an eBrief system architecture 100. The eBrief system architecture 100 may include an eBrief 102, an eBin 104, and a point of care unit 108. In some cases, the eBrief 102 may be a sanitary product with an integrated moisture sensor. The eBin 104 may be configured for disposing used sanitary products. The point of care unit 108 may be a tablet or other computing device for displaying information to caregivers.

In the eBrief system architecture 100 shown in FIG. 22, the eBrief 102 may communicate directly with the point of care unit 108. The eBin 104 may also communicate with the point of care unit 108. This configuration may allow for moisture events detected by the eBrief 102 to be transmitted to the point of care unit 108 without an intermediary device.

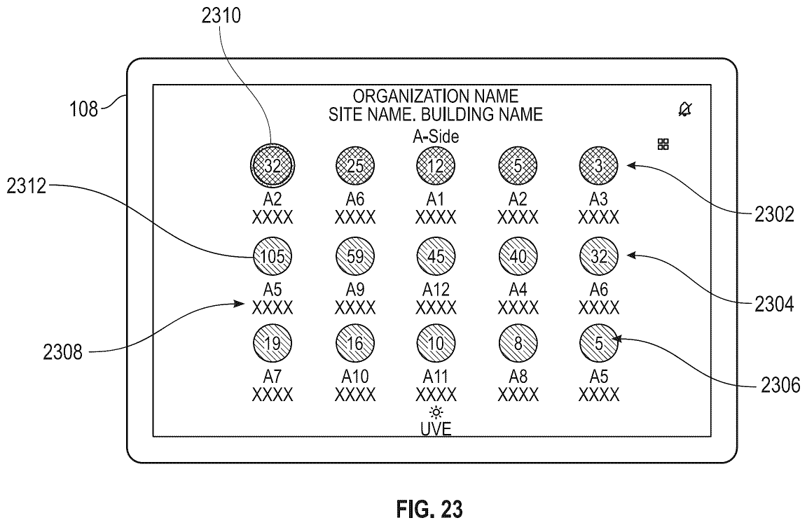

FIG. 23 depicts another embodiment of the eBrief system architecture 100. In this configuration, the eBrief system architecture 100 may include an eBrief 102, an eBin 104, a reader 106, and a point of care unit 108. The reader 106 may be positioned to receive signals from the eBrief 102 within its detection range.

In some cases, the reader 106 may communicate with the point of care unit 108, which may display status information about monitored residents. The eBrief system architecture 100 shown in FIG. 23 may enable monitoring of multiple residents simultaneously. The reader 106 may receive signals from any activated eBrief 102 within range and forward that information to the point of care unit 108. The status information may be presented using patient tiles 2312, each representing a different resident or monitored location.

In some cases, the tiles 2312 may use color coding to indicate wetness detection status. A tile 2312 displayed in a first color 2302, represented by crossed lines in the figure, may indicate that wetness has been detected for that patient. Conversely, a tile 2312 shown in a second color 2304, indicated by slanted lines, may signify that no wetness has been detected.

Each tile 2312 may include a number 2306 that provides temporal information. For tiles 2312 in the first color 2302, the number 2306 may represent the duration since wetness was initially detected. In tiles 2312 shown in the second color 2304, the number 2306 may indicate the time elapsed since the patient's last check or change.

The dashboard may also include identification information 2308 below each tile 2312. This information may specify the room and/or bed where the patient is located, allowing for quick identification and localization of care needs.

In some implementations, the display may feature a critical event indicator 2310. This indicator may be used to communicate if a patient has remained in a wet or dry state for an extended period. The system may employ various methods to draw attention to critical events 2310, such as causing the tile 2312 to pulse or having the point of care unit 108 generate audible or tactile notifications.

These visual and interactive elements of the dashboard interface may allow caregivers to quickly assess the status of multiple patients, prioritize care activities, and respond promptly to moisture events or extended periods without changes. The color-coded system, numerical indicators, and critical event alerts may work together to provide a comprehensive overview of patient status and care needs across the monitored area.

The point of care unit 108 in FIG. 23 may display a dashboard interface showing the status of various residents and their associated eBriefs 102. In some cases, the dashboard may identify each resident by a number, which may correspond to a room and/or bed where each resident being tracked is located.

The eBin 104 shown in FIG. 22 may provide a disposal location for used eBriefs while maintaining system monitoring capabilities. In some cases, the eBin 104 may be a smart bin configured to detect when a used eBrief 102 is disposed and communicate this information to the point of care unit 108.

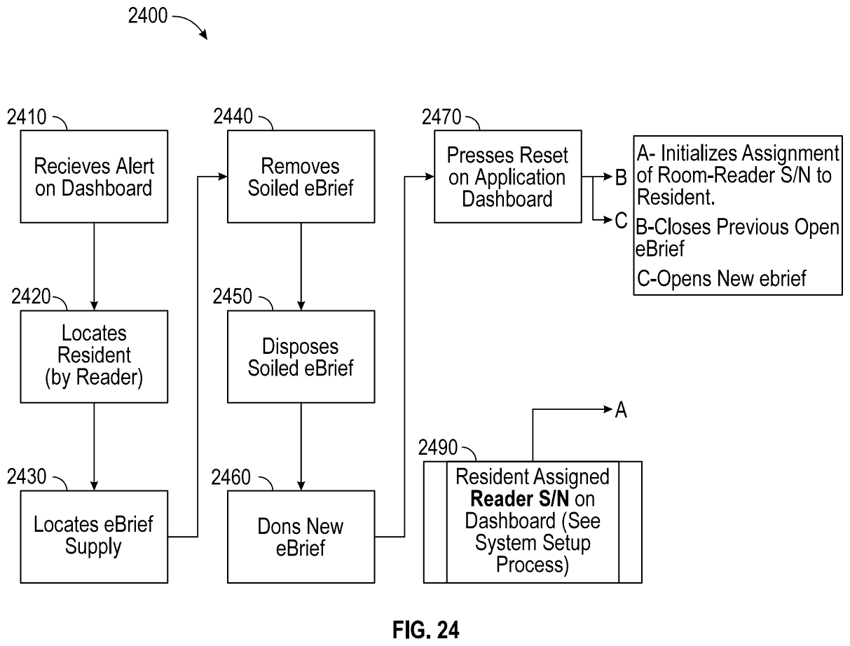

The system may implement various workflows for managing moisture detection and care processes. FIG. 24 illustrates a reader-assigned workflow 2400 in accordance with one or more embodiments. The reader-assigned workflow 2400 begins with a step 2410 where a dashboard receives a fluid detection alert. In a step 2420, a reader identifies and locates a resident associated with the tag giving the fluid detection signal. The workflow 2400 then proceeds to a step 2430, where a supply of clean eBriefs is located.

Following this, a step 2440 involves removing the soiled eBrief from the resident. The workflow 2400 then moves to a step 2450 where the soiled eBrief is disposed of. In a step 2460, the resident dons a new eBrief and tag. A step 2470 includes resetting the application dashboard for the soiled tag. The workflow 2400 concludes with steps 2480 and 2490 where the reader initializes an assignment of a new identification number to the resident's new tag, terminates the previous tag, and opens a new eBrief to be tracked.

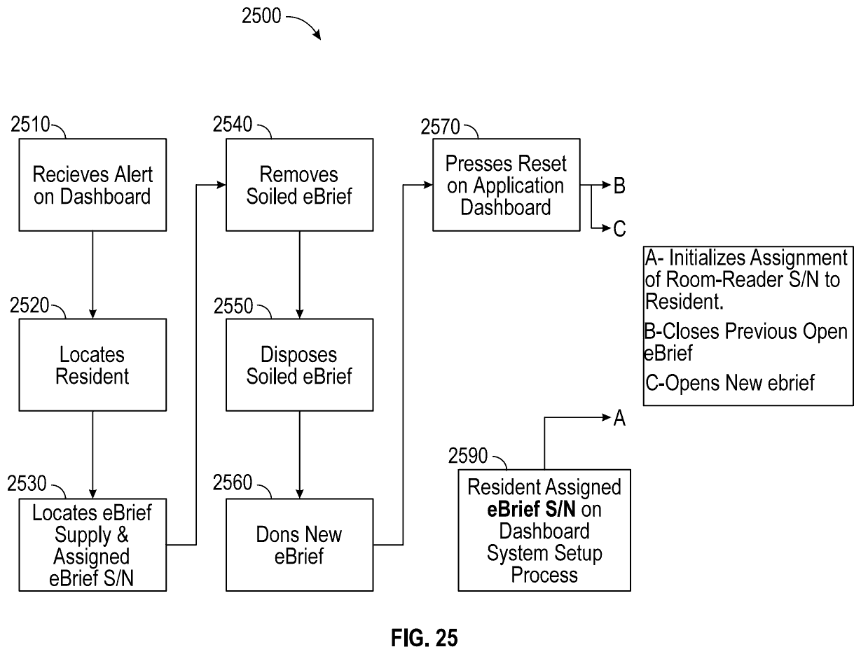

FIG. 25 depicts a tag-ID assigned workflow 2500. In this workflow, a batch of eBriefs with unique IDs may be assigned to a resident. The unique IDs may be stored in a database. A reader may detect a void event from an eBrief and forward the void event to a server. The server may match the eBrief ID to a resident and forward a message to a caretaker application. A caretaker may then locate the resident, change the resident, dispose of the soiled eBrief, don a new eBrief on the resident, and clear the alert.

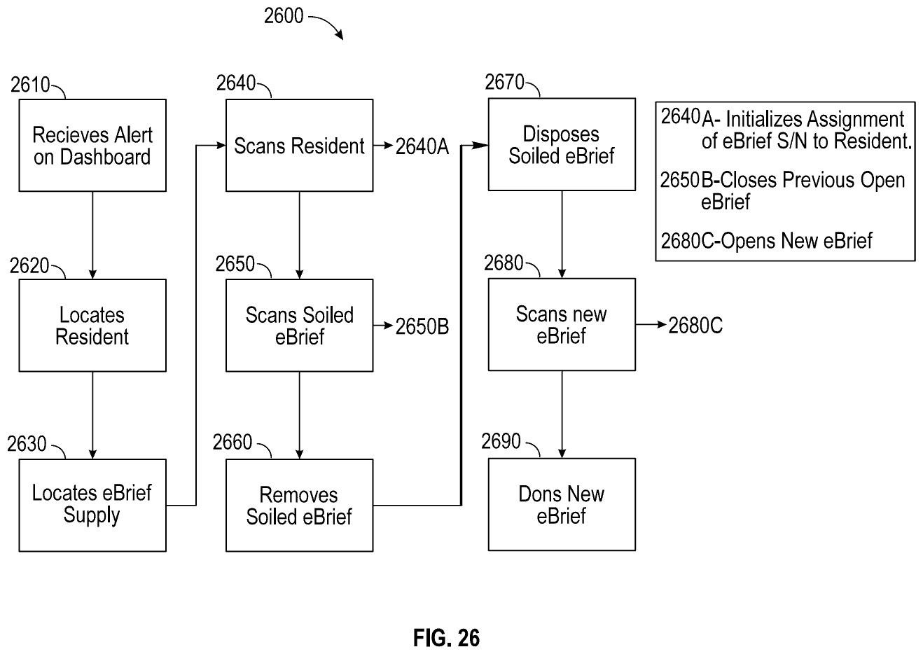

FIG. 26 illustrates a scanner assigned workflow 2600. The scanner-assigned workflow 2600 begins with a step 2610 where a dashboard receives a fluid detection alert. In a step 2620, a reader identifies and locates a resident with the tag giving the fluid detection signal. The workflow 2600 then proceeds to a step 2630, where a supply of clean eBriefs is located.

In a step 2640, the resident is scanned. The step 2640 may include initializing assignment of an eBrief to the resident in response to the scanning. A step 2650 involves scanning the soiled eBrief, which may close the previously open eBrief in the system. In a step 2660, the resident removes the soiled eBrief and tag. A step 2670 includes disposing of the soiled eBrief and tag.

The workflow 2600 continues with a step 2680 where a new eBrief and tag are scanned. This step may include opening a new eBrief to be tracked in association with the resident. The workflow 2600 concludes with a step 2690 where the resident dons the new eBrief.

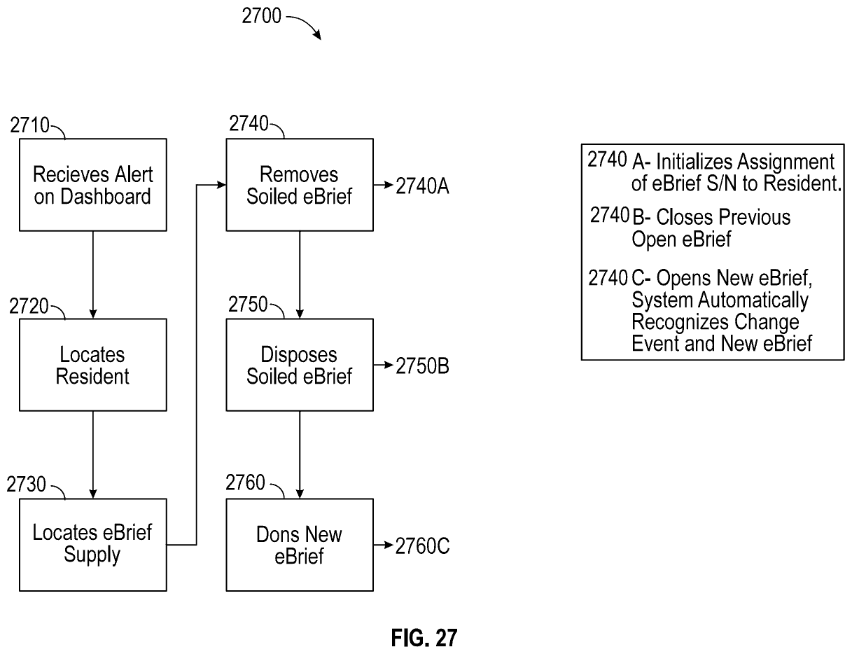

FIG. 27 depicts an auto-assigned workflow 2700. The auto-assigned workflow 2700 begins with a step 2710 where a dashboard receives a fluid detection alert. In a step 2720, a reader identifies and locates a resident with the tag giving the fluid detection signal. The workflow 2700 then proceeds to a step 2730, where a supply of clean eBriefs is located.

A step 2740 involves removing the soiled eBrief from the resident. This step may include automatically initializing assignment of an eBrief to the resident. The workflow 2700 then moves to a step 2750 where the soiled eBrief is disposed of in an eBin. This step may include automatically closing the previously open eBrief in the system.

The workflow 2700 concludes with a step 2760, where the resident dons a new eBrief and tag. This step may include automatically opening a new eBrief to be tracked in association with the resident.

In some cases, the supply of eBriefs may be stored in a first eBin and the soiled eBrief may be disposed of in a second eBin. The automatic initialization of the new eBrief assignment may occur when the first eBin detects that a new eBrief has been removed from the supply. The automatic closing of the previously open eBrief may occur when the second eBin detects the soiled eBrief has been disposed of. The automatic opening of a new eBrief to be tracked may occur when the first eBin detects that a new eBrief has been removed from the supply and when the second eBin detects the soiled eBrief has been disposed of.

These various workflows may provide flexibility in different care scenarios. The reader-assigned workflow may be suitable for facilities with fixed reader locations. The tag-ID assigned workflow may allow for more mobility of residents within a facility. The scanner-assigned workflow may provide precise control over eBrief assignments. The auto-assigned workflow may reduce manual steps and increase efficiency in high-volume care environments.

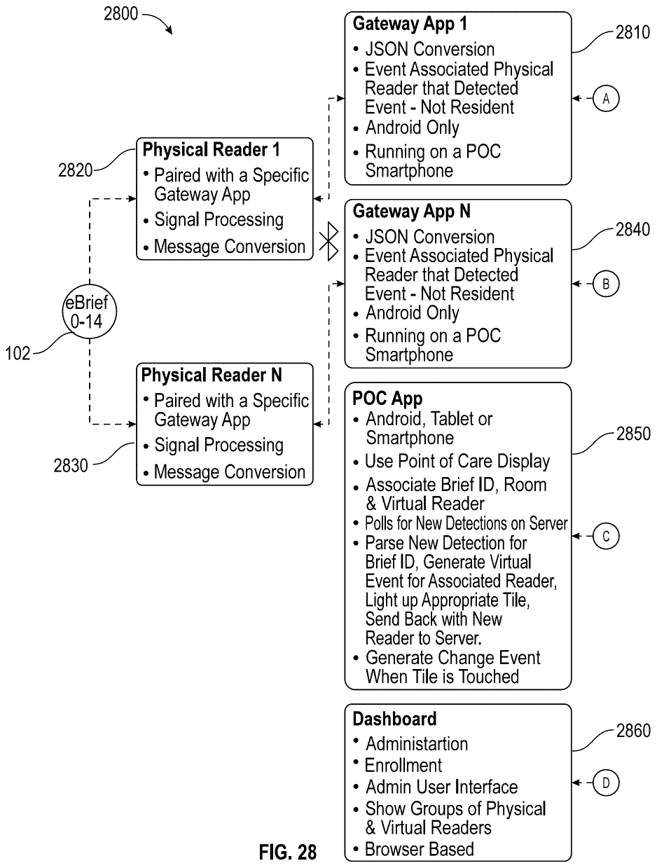

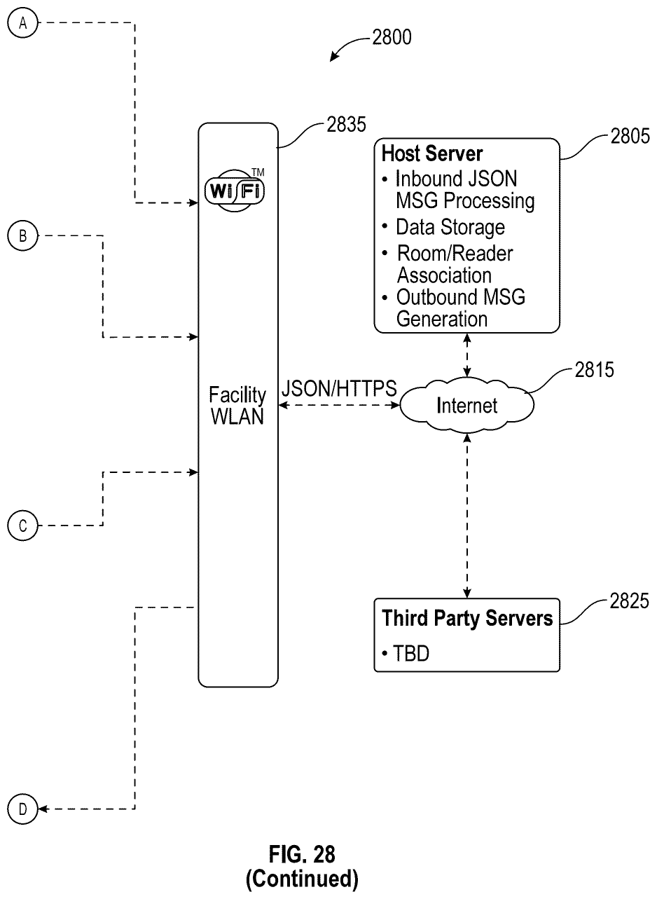

FIG. 28A and FIG. 28B illustrate dataflow and connectivity architectures 2800 for the moisture detection system. FIG. 28A depicts a high-level overview of the system components and their interactions, while FIG. 28B provides a more detailed view of the network architecture.

Referring to FIG. 28A, the dataflow architecture includes a physical reader 2900 that may detect signals from an eBrief 1027. The physical reader 2900 may communicate with a gateway app 2940, which processes and forwards the data. The gateway app 2940 may be part of a first gateway app 2810 or an Nth gateway app 2840, depending on the number of gateways in the system. The processed data may then be sent to a dashboard 2860, which may be part of a point of care (POC) app 2850.

FIG. 28B illustrates a more detailed network architecture for data communication. The architecture includes a facility WLAN 2835 that may provide wireless network connectivity within a care facility. The facility WLAN 2835 may connect to a host server 2805 and communicate with the internet 2815 using secure protocols such as JSON/HTTPS.

The host server 2805 may process inbound JSON messages, handle data storage, manage room/reader associations, and generate outbound messages. The facility WLAN 2835 may receive inputs from multiple sources through wireless connections.

The internet 2815 may enable communication between the facility WLAN 2835 and third-party server(s) 2825. This connection may allow for integration with external systems or services that may enhance the functionality of the moisture detection system.

In some cases, the system may use image processing and artificial intelligence/machine learning (AI/ML) methods to detect void events and changes. For example, cameras or other sensors connected to the facility WLAN 2835 may capture images or data that can be processed by AI algorithms on the host server 2805 or third-party server(s) 2825 to identify potential moisture events or resident behavior patterns.

The system may also track inventory and automatically reorder supplies. The host server 2805 may maintain a database of eBrief inventory levels. When supplies run low, the host server 2805 may generate an order request that can be sent through the internet 2815 to third-party server(s) 2825 associated with suppliers.

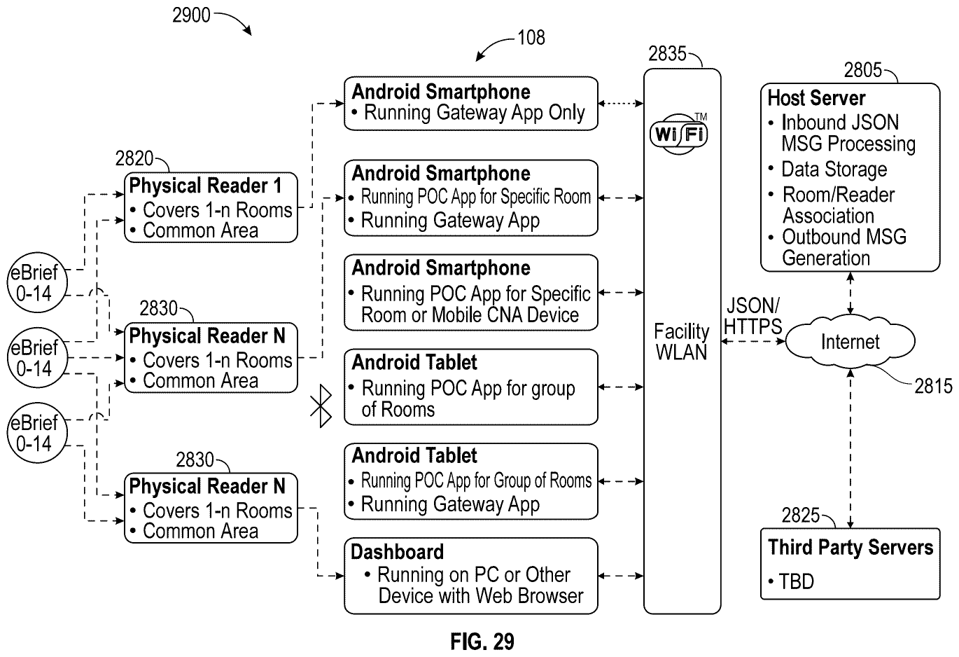

FIG. 29 may provide additional details on the data flow 2800 within the system. The data flow 2800 may illustrate how moisture event information moves from the eBrief 1027 through the physical reader 2900, gateway app 2940, and ultimately to the dashboard 2860 where care staff can view and respond to alerts.

This architecture may allow for efficient data flow between the facility components and external systems through the internet 2815, while the host server 2805 manages internal data processing and storage functions. The system may provide real-time monitoring of moisture events, inventory management, and integration with external services to enhance resident care and operational efficiency.

The moisture detection system may include a tag 200. The tag 200 may comprise an integrated circuit 214, a capacitor 3104, and a tag ID 3106. In some cases, the integrated circuit 214 may be positioned centrally on the tag 200. The capacitor 3104 may be located adjacent to the integrated circuit 214. The tag ID 3106 may be distributed across portions of the tag 200 surface.

In some cases, the integrated circuit 214 may control the operation of the tag 200. The capacitor 3104 may store electrical energy to power the tag 200. The tag ID 3106 may provide a unique identifier for each tag 200.

The integrated circuit 214, capacitor 3104, and tag ID 3106 may be arranged in a planar configuration on the tag 200. This arrangement may allow for a compact design while maintaining functionality.

In some cases, the integrated circuit 214 may process signals from moisture detection. The capacitor 3104 may supply power for signal transmission. The tag ID 3106 may be included in transmitted signals to identify the specific tag 200.

The components of the tag 200 may work together to enable moisture detection and signal transmission. When moisture is detected, the integrated circuit 214 may activate, drawing power from the capacitor 3104. The integrated circuit 214 may then generate a signal including the tag ID 3106, which may be transmitted to a reader.

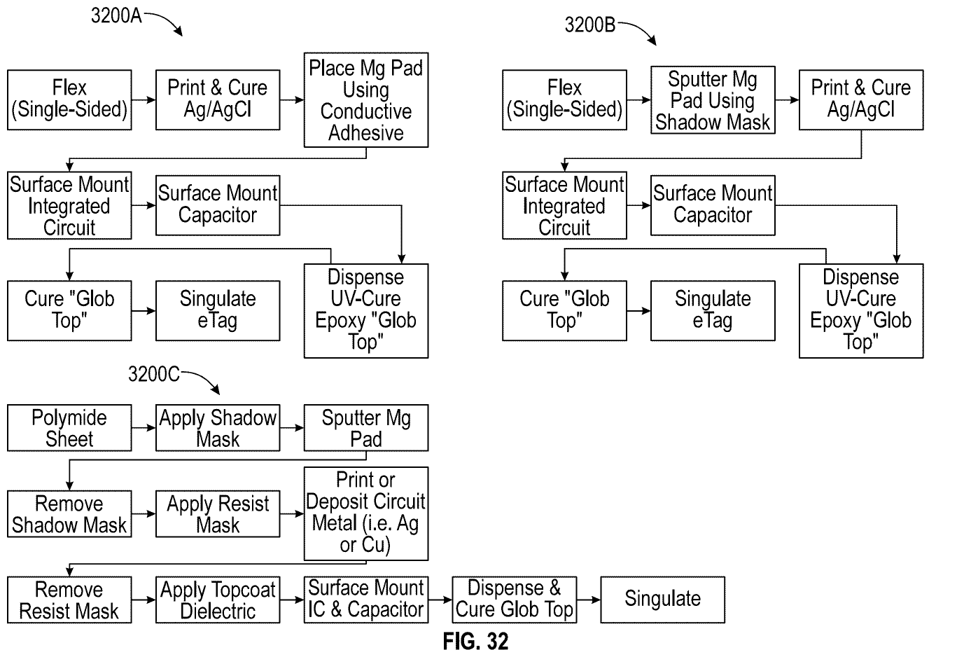

FIG. 32 illustrates various manufacturing processes for the eBrief tag. The figure depicts three distinct assembly processes: a standard eBrief tag FPC assembly process, an eBrief tag FPC assembly process with sputtered magnesium, and an eTag assembly process with sputtered magnesium.

In the standard eBrief tag FPC assembly process, the tag may be manufactured using conventional flexible printed circuit (FPC) techniques. This process may involve printing conductive traces on a flexible substrate, attaching electronic components, and applying protective coatings.

The eBrief tag FPC assembly process with sputtered magnesium introduces an additional step. In this process, magnesium may be deposited onto the tag surface using a sputtering technique. Sputtering involves bombarding a magnesium target with high-energy particles, causing magnesium atoms to be ejected and deposited onto the tag substrate in a thin, uniform layer. This process may allow for precise control over the thickness and coverage of the magnesium layer.

The eTag assembly process with sputtered magnesium may be similar to the eBrief tag process with sputtered magnesium, but may be optimized for the specific requirements of the eTag design. This process may involve different substrate materials, component layouts, or sputtering parameters tailored to the eTag's form factor and functionality.

The inclusion of magnesium in these processes may provide several potential benefits. Magnesium may serve as one of the electrodes in the moisture detection system, offering a galvanic potential when paired with another dissimilar metal. This galvanic pair may generate a small electrical current when exposed to moisture, enabling the tag to detect and signal the presence of fluid without requiring an external power source.

In some cases, the eBrief tag may be manufactured using alternative methods for incorporating magnesium. These methods may include stamping a magnesium foil onto the tag surface or printing a custom magnesium metallic ink or nano-ink. Each of these manufacturing techniques may offer different advantages in terms of cost, scalability, and performance characteristics of the resulting tags.

The choice of manufacturing process may depend on factors such as production volume, desired tag performance, and cost considerations. The sputtering process may offer high precision and uniformity, while stamping or printing processes may be more suitable for high-volume production or specific tag designs.

These various workflows may provide flexibility in different care scenarios. The reader-assigned workflow may be suitable for facilities with fixed reader locations. The tag-ID assigned workflow may allow for more mobility of residents within a facility. The scanner-assigned workflow may provide precise control over eBrief assignments. The auto-assigned workflow may reduce manual steps and increase efficiency in high-volume care environments.

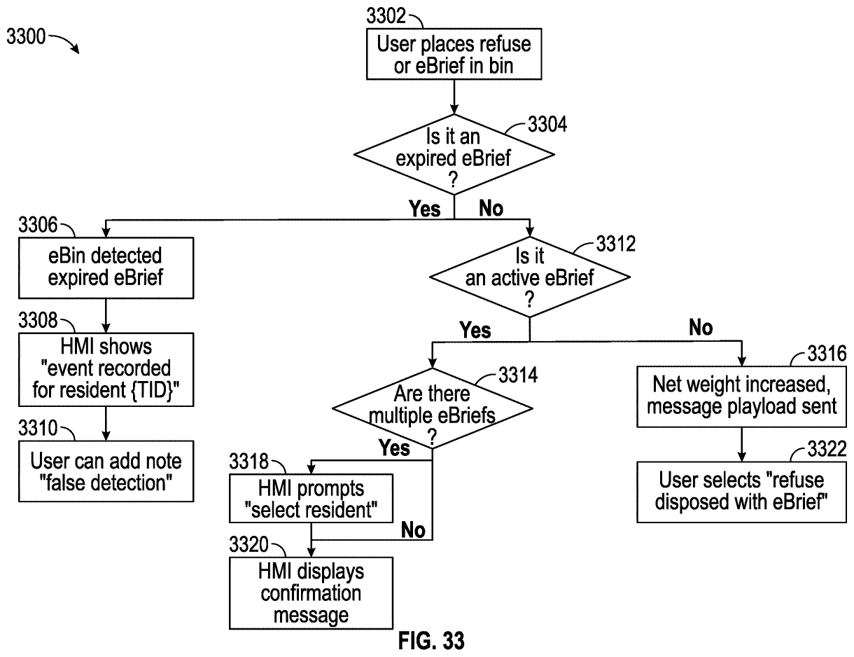

FIG. 33 illustrates a method 3300 for handling refuse or eBrief disposal in a bin. The method 3300 may enhance the overall functionality of the moisture detection system by providing a systematic approach to managing disposed items and tracking eBrief usage.

The method 3300 begins with a step 3302, where a user places refuse or an eBrief in the bin. Following this, a step 3304 involves determining whether the item is an expired eBrief.

If the item is identified as an expired eBrief, the method 3300 proceeds to a step 3306, where the eBin detects the expired eBrief. In some cases, this detection may be based on the eBrief's unique identifier or lack of active signal. The method 3300 then continues to a step 3308, where the human-machine interface (HMI) displays a message indicating “event recorded for resident (TID)”. This step may allow for accurate tracking of eBrief usage and disposal. Following this, a step 3310 allows the user to add a note, such as “false detection”, providing an opportunity for manual correction of any system errors.

If the item is not an expired eBrief, the method 3300 moves to a step 3312, which determines whether the item is an active eBrief. This step may involve detecting a signal from the eBrief or reading its unique identifier.

In cases where the item is not an active eBrief, the method 3300 proceeds to a step 3316. During step 3316, the net weight of the bin may be increased, and a message payload may be sent. This step may allow for tracking of general refuse disposal and bin capacity.

If the item is identified as an active eBrief, the method 3300 moves to a step 3314, which determines whether there are multiple eBriefs being disposed of simultaneously. This step may help in accurately tracking the disposal of multiple items.

In cases where multiple eBriefs are detected, the method 3300 proceeds to a step 3318. During step 3318, the HMI may prompt the user to “Select Resident”, allowing for proper association of the disposed eBriefs with the correct residents.

If there are not multiple eBriefs, the method 3300 continues to a step 3320. During step 3320, the HMI may display a confirmation message, potentially indicating successful recording of the eBrief disposal.

Following the confirmation message, the method 3300 may proceed to a step 3322. During step 3322, the user may have the option to select “refuse disposed with eBrief”. This step may allow for accurate tracking of instances where additional refuse is disposed of along with an eBrief.

The method 3300 demonstrates a decision-making process for handling different types of refuse and eBrief disposal scenarios. By providing appropriate system responses and user interactions based on the type of item being disposed, the method 3300 may enhance the accuracy of eBrief usage tracking and improve overall waste management within the moisture detection system.

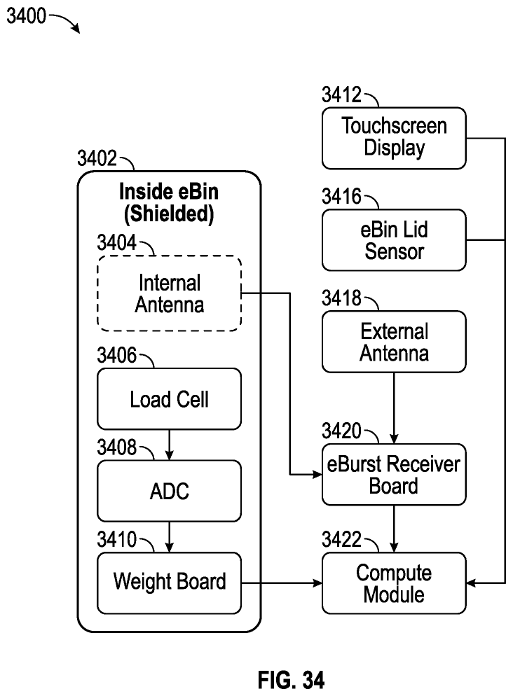

FIG. 34 illustrates a system architecture 3400 for a smart bin monitoring system. The system architecture 3400 may include components arranged within and outside an inside bin 3402.

Within the inside bin 3402, an internal antenna 3404 may be positioned to detect signals. A load cell 3406 may connect to an analog-digital converter 3408, which in turn may connect to a weight board 3410. These components may work together to measure and process weight data from items placed in the bin.

The system architecture 3400 may include a touchscreen display 3412 for user interface purposes. A bin lid sensor 3416 may monitor the status of the bin lid. An external antenna 3418 may be positioned outside the inside bin 3402 to receive signals.

The system architecture 3400 may further include a receiver board 3420 that processes signals from both the internal antenna 3404 and external antenna 3418. A compute module 3422 may receive inputs from the weight board 3410, touchscreen display 3412, bin lid sensor 3416, and receiver board 3420. The compute module 3422 may process these various inputs to monitor and track bin usage and contents.

The components may be arranged to enable detection of signals both inside and outside the bin while measuring weight and allowing user interaction through the touchscreen display 3412. In some cases, the system architecture 3400 facilitates monitoring of disposed items through weight measurement and signal detection capabilities.

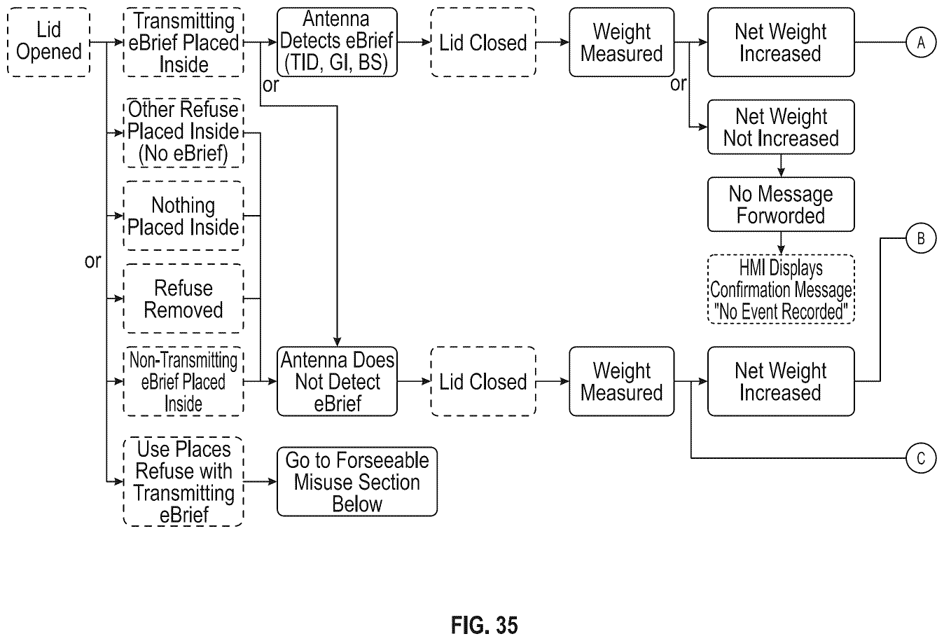

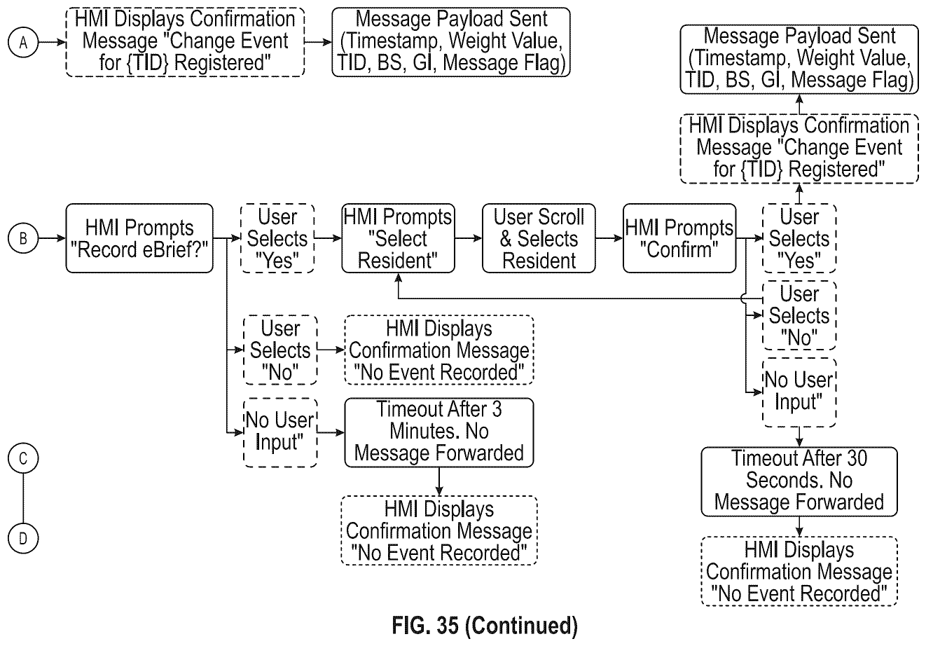

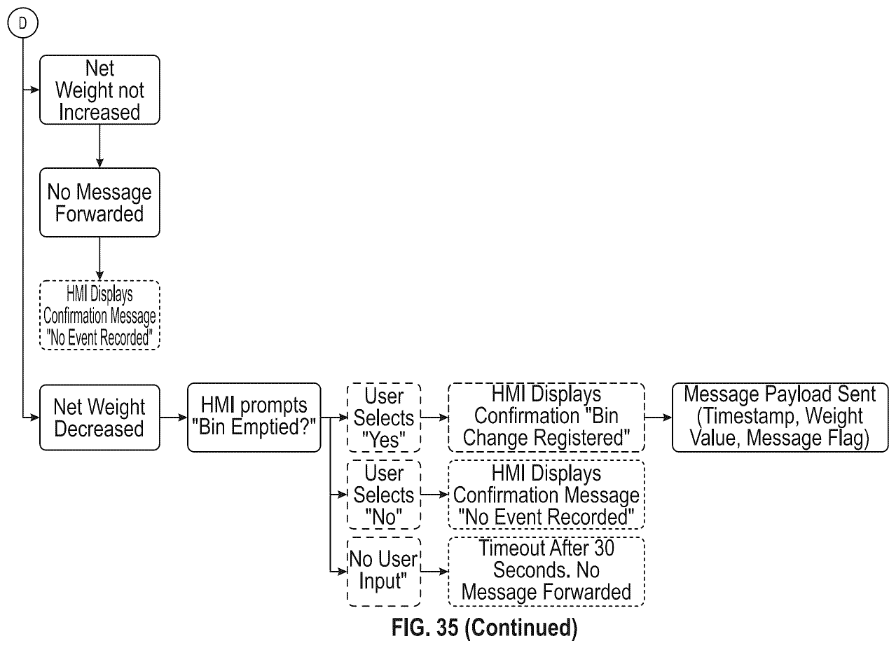

FIG. 35 and FIG. 35 (Continued) may illustrate a workflow for handling refuse or eBrief disposal in a bin system. The workflow may begin when a user opens the bin lid and places an item in the bin.

Upon lid opening, the system may take a weight measurement. The workflow may then proceed to determine if there has been a net weight increase. If no weight increase is detected, the system may follow a path indicating no message is forwarded.

In cases where a weight increase is detected, the workflow may branch into parallel paths. One path may lead to determining whether an antenna detects an eBrief, while another path may determine if the lid is closed. These paths may converge at a point where the system determines whether the net weight has increased.

The workflow may include additional decision points for processing multiple scenarios. For example, the system may handle cases where multiple eBriefs are present with matching or non-matching identification numbers. At various stages, the workflow may provide appropriate human-machine interface (HMI) feedback based on the detected conditions.

The system may also include steps for handling edge cases and potential misuse scenarios, such as when refuse is placed in the bin along with an eBrief. The workflow may incorporate weight tracking, event data transmission with timestamps, and processing of various input combinations from the lid sensor, weight sensor, and antenna.

Depending on the detected conditions, the workflow may conclude with different endpoints. Some paths may lead to message forwarding, while others may result in HMI feedback to users. The system may process and respond to different input combinations in a systematic manner throughout the workflow.

In cases where the lid is opened but no weight increase is detected, the workflow may include steps to determine if the bin has been emptied. This may involve prompting the user through the HMI and checking for a net weight decrease.

The workflow may also include timing considerations, with a timeout period shown for certain steps. This may help ensure the system continues to function even if expected inputs are not received within a specified timeframe.

Throughout the workflow, the system may generate and send message payloads containing relevant data such as timestamps, weight values, and other parameters. These messages may be used for tracking and reporting purposes within the larger moisture detection system.

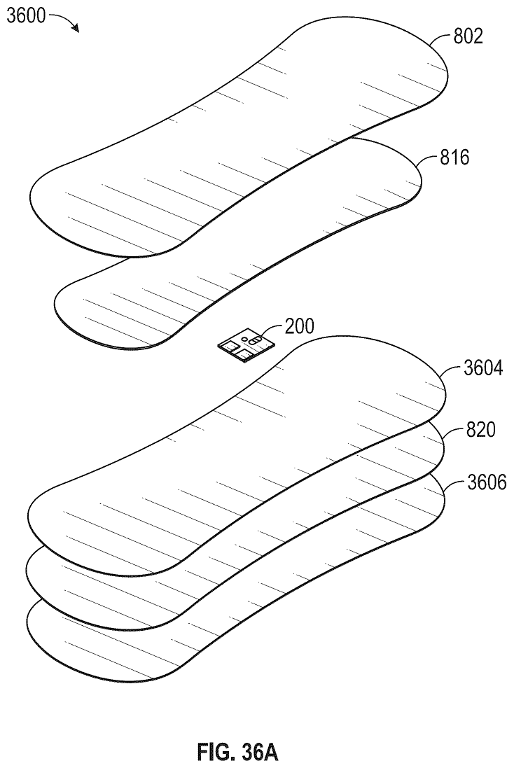

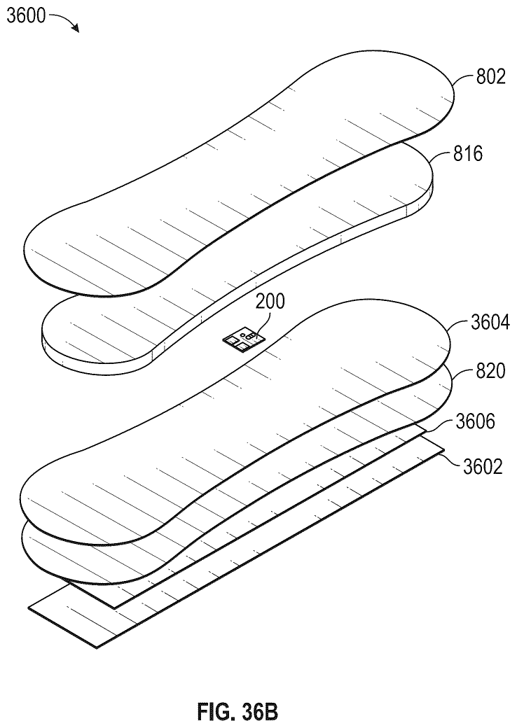

FIG. 36A illustrates an exploded view of a sensor insert 3600 embodiment showing the layered construction of the device. The sensor insert 3600 may comprise multiple layers arranged in a stacked configuration. From top to bottom, these layers may include a non-woven material 802, followed by a super absorbent polymer 816. A tag 200 may be positioned between super absorbent polymer 816 and an inner adhesive layer 3604 adhering the tag 200 between the super absorbent polymer 816 and the back sheet 820. The final layer of the sensor insert 3600 may be an outer adhesive layer 3606 configured to adhere the insert to a personal hygiene product.

FIG. 36B illustrates another exploded view of another sensor insert 3600 embodiment showing an alternative layered construction of the device. The view displays multiple layers arranged in a stacked configuration from top to bottom. The non-woven material 802 may form the uppermost layer of the assembly. Below this, the super absorbent polymer 816 may be positioned, followed by tag 200. An inner adhesive layer 3604 may be included to secure the components together. The back sheet 820 may be disposed between the inner adhesive layer 3604 and an outer adhesive layer 3606 forming the bottom layer of the assembly. A release liner 3602 may be temporarily adhered to the outer adhesive layer 3606. Once the sensor insert 3600 is ready to be inserted into the hygiene product, the release liner 3602 may be removed, exposing the outer adhesive layer 3606.

This exploded views may demonstrate how the various layers are arranged relative to each other in a vertical stack. The separation between layers may illustrate the order of assembly and the spatial relationship between components. The tag 200 may be positioned between the super absorbent polymer 816 and the adhesive 808, potentially allowing it to detect moisture while remaining secured within the structure.

In some aspects, the different configurations shown in FIG. 36A and FIG. 36B may provide flexibility in manufacturing and assembly processes, allowing for optimization of moisture detection sensitivity and overall product performance based on specific application requirements.

FIG. 36A illustrates an exploded view of a sensor insert 3600 embodiment showing the layered construction of the device. The sensor insert 3600 may comprise multiple layers arranged in a stacked configuration. From top to bottom, these layers may include a non-woven material 802, followed by a super absorbent polymer 816. A tag 200 may be positioned between super absorbent polymer 816 and an inner adhesive layer 3604 adhering the tag 200 between the super absorbent polymer 816 and the back sheet 820. The final layer of the sensor insert 3600 may be an outer adhesive layer 3606 configured to adhere the insert to a personal hygiene product.

FIG. 36B illustrates another exploded view of another sensor insert 3600 embodiment showing an alternative layered construction of the device. The view displays multiple layers arranged in a stacked configuration from top to bottom. The non-woven material 802 may form the uppermost layer of the assembly. Below this, the super absorbent polymer 816 may be positioned, followed by tag 200. An inner adhesive layer 3604 may be included to secure the components together. The back sheet 820 may be disposed between the inner adhesive layer 3604 and an outer adhesive layer 3606 forming the bottom layer of the assembly. A release liner 3602 may be temporarily adhered to the outer adhesive layer 3606. Once the sensor insert 3600 is ready to be inserted into the hygiene product, the release liner 3602 may be removed, exposing the outer adhesive layer 3606.

In some aspects, these exploded views may demonstrate how the various layers are arranged relative to each other in a vertical stack. The separation between layers may illustrate the order of assembly and the spatial relationship between components. The tag 200 may be positioned between the super absorbent polymer 816 and the adhesive layers, potentially allowing it to detect moisture while remaining secured within the structure.

The different configurations shown in FIG. 36A and FIG. 36B may provide flexibility in manufacturing and assembly processes. In some cases, this flexibility may allow for optimization of moisture detection sensitivity and overall product performance based on specific application requirements. The placement of the tag 200 and the arrangement of the adhesive layers may be adjusted to achieve desired performance characteristics in different use scenarios.

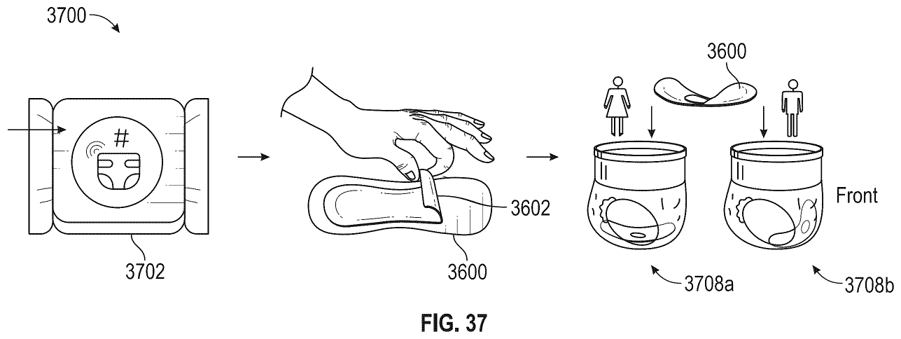

FIG. 37 illustrates an application method 3700 for a sensor insert 3600. The figure may show a sensor insert package 3702 containing one or more sensor inserts 3600. A sensor insert 3600 may be removed from the sensor insert package 3702. The figure may depict a release liner being removed from the sensor insert 3600. Using the outer adhesive layer 3606, the sensor insert may then be adhered to the inside of a personal hygiene product.

The placement of the sensor insert 3600 may vary depending on whether the patient is male or female. For male patients, the insert may be placed toward the front 3708b of the hygiene product. For female patients, the sensor insert 3600 may be adhered to the middle 3708a of the hygiene product.

The application method 3700 may demonstrate how sensor inserts can be individually removed from the sensor insert package 3702 for use. The sensor insert package 3702 may be configured to store and protect multiple sensor inserts prior to their removal and application. In some cases, the sensor inserts 3600 may be arranged in a stacked configuration within the sensor insert package 3702, potentially allowing for organized storage and easy access when needed.

This method may provide a systematic approach for applying sensor inserts to hygiene products, potentially enabling customization based on patient gender and allowing for efficient integration of moisture detection capabilities into existing hygiene product designs.

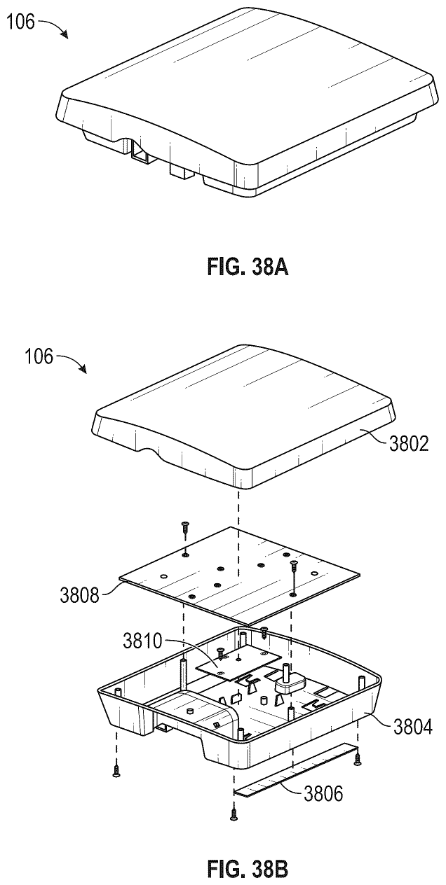

FIG. 38A and FIG. 38B may illustrate an exploded view of one or more embodiments of a reader 106. The reader 106 may comprise a front reader enclosure 3802 and a back reader enclosure 3804. The front reader enclosure 3802 and back reader enclosure 3804 may form a housing for internal components when assembled together.

The reader 106 may include an antenna assembly 3808 positioned within the enclosure. A connector assembly 3810 may also be housed within the enclosure. In some cases, the reader 106 may include a room label 3806 that can be attached to identify the location where the reader is installed.

The exploded view may show how the components of the reader 106 are arranged for assembly, with the front reader enclosure 3802 and back reader enclosure 3804 designed to enclose and protect the antenna assembly 3808 and connector assembly 3810. The room label 3806 may be applied to the exterior of the assembled enclosure.

In some aspects, this configuration may allow for easy assembly and maintenance of the reader 106. The modular design may enable components to be replaced or upgraded individually if needed. The use of separate front and back enclosures may provide flexibility in mounting options and may facilitate access to internal components for servicing or modifications.

The antenna assembly 3808 may be positioned to optimize signal reception from moisture detection tags within its range. The connector assembly 3810 may provide interfaces for power and data connections, potentially allowing the reader 106 to communicate with other components of the moisture detection system.

The room label 3806 may serve multiple purposes beyond simple identification. It may contain information such as a unique identifier for the reader, which could be used in system setup and configuration. In some implementations, the room label 3806 may include machine-readable elements like barcodes or QR codes to facilitate automated inventory and maintenance tracking.

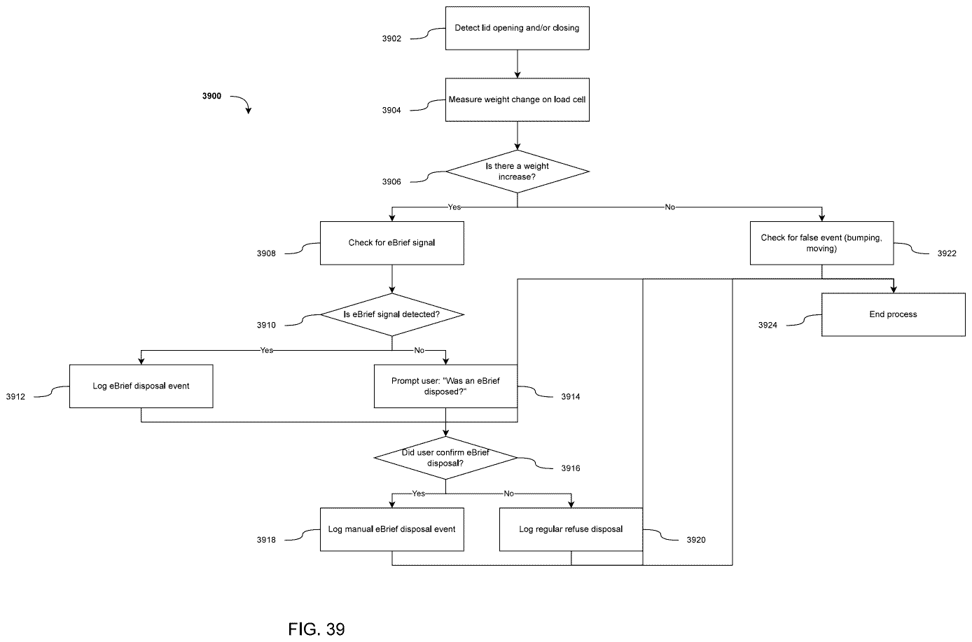

FIG. 39 illustrates a flowchart depicting a method for handling refuse or eBrief disposal in a bin. The process may begin with detecting the opening and/or closing of the bin lid, followed by measuring any weight change on a load cell. The flowchart may then proceed to a decision point to determine if there is a weight increase.

If a weight increase is detected, the process may check for an eBrief signal. In cases where an eBrief signal is detected, the system may log an eBrief disposal event. The system may also measure the weight change to determine the fluid volume of waste disposed of with the eBrief. Based on this fluid volume measurement, the system may determine a hydration state of the patient. If no eBrief signal is detected, the system may prompt the user to confirm if an eBrief was disposed. Based on the user's response, the process may either log a manual eBrief disposal event or log a regular refuse disposal.

In scenarios where no weight increase is detected, the process may move to check for a false event, such as the bin being bumped or moved without an actual disposal occurring.

The flowchart may incorporate multiple decision points and user interactions to accurately categorize disposal events. It may include steps for automated detection of eBrief signals and manual confirmation when necessary. This approach may allow for differentiation between eBrief and regular refuse disposal, utilizing both automated sensing and user input.

The method depicted in FIG. 39 may provide a structured approach to logging different types of disposal events, which may be useful for waste management and tracking purposes within the larger moisture detection system. The process may account for various scenarios, including potential false events, to enhance the accuracy of disposal event logging. The fluid volume measurements obtained through weight change analysis may further enable healthcare providers to monitor patient hydration levels over time, potentially identifying dehydration risks or other health concerns based on voiding patterns. In a further embodiment, the system may also track the fluid intake of the patient and determine a hydration state based off both the fluid intake and the fluid volume of the waste, providing a more comprehensive assessment of the patient's hydration status.

FIG. 39 illustrates a flowchart depicting a method for handling refuse or eBrief disposal in a bin. The process may begin with a step 4000 of detecting the opening and/or closing of the bin lid, followed by a step 4002 of measuring any weight change on a load cell. The flowchart may then proceed to a decision point 4004 to determine if there is a weight increase.

If a weight increase is detected, the process may move to a step 4006 of checking for an eBrief signal. In cases where an eBrief signal is detected, the system may proceed to a step 4008 of logging an eBrief disposal event. The system may then move to a step 4010 of measuring the weight change to determine the fluid volume of waste disposed of with the eBrief. Based on this fluid volume measurement, the system may proceed to a step 4012 of determining a hydration state of the patient.

If no eBrief signal is detected at step 4006, the system may move to a step 4014 of prompting the user to confirm if an eBrief was disposed. Based on the user's response, the process may branch to either a step 4016 of logging a manual eBrief disposal event or a step 4018 of logging a regular refuse disposal.

In scenarios where no weight increase is detected at decision point 4004, the process may move to a step 4020 of checking for a false event, such as the bin being bumped or moved without an actual disposal occurring.

The flowchart may incorporate multiple decision points and user interactions to accurately categorize disposal events. It may include steps for automated detection of eBrief signals and manual confirmation when necessary. This approach may allow for differentiation between eBrief and regular refuse disposal, utilizing both automated sensing and user input.

In some cases, the method may include a step 4022 of tracking fluid intake of the patient. The system may then proceed to a step 4024 of determining a comprehensive hydration state based on both the fluid intake and the fluid volume of the waste, potentially providing a more complete assessment of the patient's hydration status.

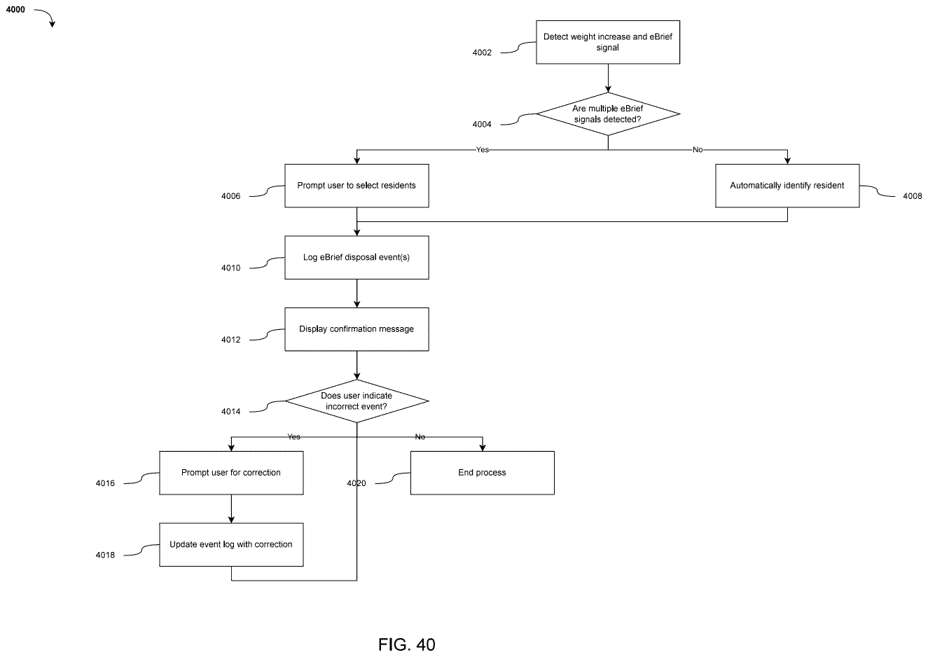

FIG. 40 illustrates a flowchart depicting a method for handling multiple eBrief disposals and user corrections. The process may begin with a step where the system detects a weight increase and eBrief signal. The flowchart may then proceed to a decision point to determine if multiple eBrief signals are detected.

In cases where multiple signals are detected, the process may prompt the user to select residents associated with the disposed eBriefs. If a single signal is detected, the process may automatically identify the resident. Both paths may then converge at a step where the eBrief disposal event(s) are logged.

The process may continue with a step where a confirmation message is displayed. Following this, another decision point may check if the user indicates an incorrect event. If the user indicates an error, the process may prompt for correction and update the event log accordingly. If no error is indicated, or after any corrections are made, the process may conclude.

This flowchart may demonstrate a sequence for managing single or multiple eBrief disposals, incorporating user verification and correction capabilities. The process may allow for both automated and user-assisted identification of residents, and may include a mechanism for users to correct any logging errors. This approach may enhance the accuracy of eBrief disposal records and provide flexibility in handling various disposal scenarios.

In some implementations, the system may use machine learning algorithms to improve the accuracy of automatic resident identification over time based on disposal patterns and user corrections. The flowchart may also be adapted to include additional steps for tracking inventory, generating alerts for low supplies, or integrating with other healthcare management systems.

FIG. 40 illustrates a flowchart depicting a method for handling multiple eBrief disposals and user corrections. The process begins with step 4000 where the system detects a weight increase and eBrief signal. The flowchart then proceeds to a decision point 4001 to determine if multiple eBrief signals are detected.

If multiple signals are detected (Yes branch from 4001), the process moves to step 4002 where the user is prompted to select residents associated with the disposed eBriefs. If a single signal is detected (No branch from 4001), the process proceeds to step 4003 where the resident is automatically identified by the system.

Both paths then converge at step 4004 where the eBrief disposal event(s) are logged in the system. The process continues to step 4005 where a confirmation message is displayed to the user.

Following the confirmation message, the flowchart moves to another decision point 4006 which checks if the user indicates an incorrect event. If the user indicates an error (Yes branch from 4006), the process proceeds to step 4007 where the user is prompted for correction. This is followed by step 4008 where the event log is updated with the correction provided by the user.

If no error is indicated (No branch from 4006), or after any corrections are made in step 4008, the process concludes at step 4009.

This workflow may provide a systematic approach for managing single or multiple eBrief disposals, incorporating user verification and correction capabilities. The process may allow for both automated and user-assisted identification of residents, and may include a mechanism for users to correct any logging errors, potentially enhancing the accuracy of eBrief disposal records.

In the embodiment illustrated in FIG. 1, the sanitary product having the electronic sensor is referred to as an “eBrief” 102. The use of eBrief 102 may be used to describe the sanitary product having the electronic sensor or tag 200 and is not limited to only embodiments utilizing briefs. For example, the sanitary product may be a diaper, pullup, sanitary pad, liner, wipe, or other sanitary product. The reader 106, also referred to as a gateway, may be any device configured to detect a signal from the brief and relay a message to a server, which may in turn communicate the message to the point of care unit 108. The point of care unit 108 may be any electronic device comprising a display that communicates moisture detection to a user from the message received by the reader 106. As illustrated in FIG. 1, the reader 106 senses an activated eBrief 102 and may forward a message to a database server (not shown), that may forward the message to the point of care unit 108. For example, the message may be forwarded to a nursing station and/or a caretaker application to alert a user that moisture was detected. The alert may be a visual and/or auditory alert.

The sensor, referred interchangeably as a tag 200 or eBrief Tag, may comprise an RF transmitter, an integrated circuit 214, a capacitor 210, and a power supply which utilizes two dissimilar metals. When moisture contacts the metal pads and is in electrical communication with a fluid electrolyte, a voltage is created and current is sourced from the metal pads, according to their galvanic potential, and powers an RF transmitter. The RF transmitter transmits a signal which is controlled by the integrated circuit 214. The signal may be detected by the reader 106.

One embodiment of the tag 200 is illustrated in FIG. 2. In one embodiment, the tag 200 may comprise an identifier, for example a number, code, or other label, that may be used by the system to identify the tag 200.

In one embodiment, the tag 200 may be integrated with a sticker substrate and adhesive, which may be peeled from a roll or sheet and adhered to the inside of the sanitary product. It is contemplated that this may enable staff caretakers to have more flexibility in the use application of the sensor in cases where various sanitary products may be desired for use.

In some embodiments, the tag 200 may be integrated into a sticker with its electrode facing the surface which the sticker would be applied to, also referred to as “face-down.” An adhesive layer may hold the tag 200 and when pressed onto a desired surface of the hygiene product, may secure the tag 200 to the surface.

In an embodiment, a sticker is created with an absorbent layer over the surface of the tag 200. In this embodiment the moisture sensing electrodes are facing a non-woven material toward the wearer's skin, also referred to as “face-up.” The sensor may be secured between an adhesive layer and the non-woven layer. When moisture saturates the sanitary product it ultimately migrates to the non-woven material and comes into contact with the electrodes of the sensor circuit.

In some embodiments, the eBrief 102 may utilize standard sanitary products. For example, the sanitary products may be off-the-shelf products, such as pullups or briefs that may be combined with the sensor.

The sensors may be integrated into various locations a cross-section of the sanitary product. In some embodiments, the sensor itself may also comprise various material layers to assist in capturing and retaining fluid for activation.

One example of a method for integrating the sticker comprising the tag 200 with standard sanitary products is discussed with reference to FIG. 3.

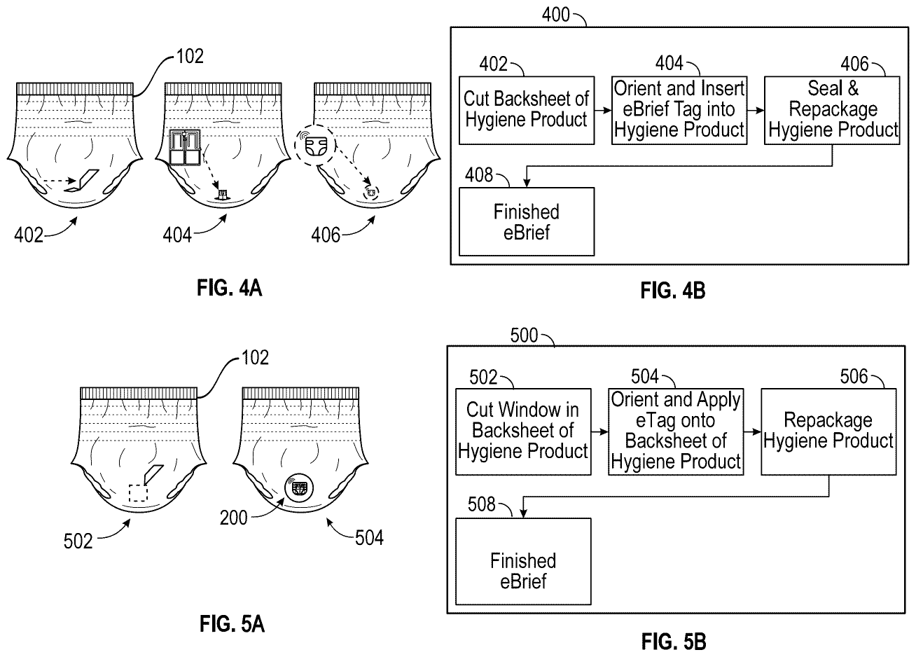

FIG. 4 illustrates one embodiment of integrating the sensor into an existing brief by cutting a portion of the brief, inserting the tag 200, and sealing the brief.

Another example of integrating the tag 200 into an existing brief is illustrated in FIG. 5. As illustrated in FIG. 5, in some embodiments a window may be cut from a portion of the brief and the tag 200 may be affixed to the brief to cover the window.

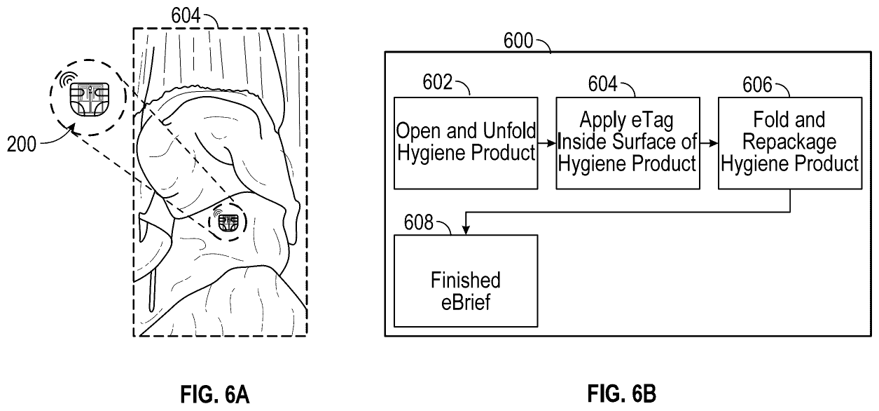

Still another embodiment of integrating the tag 200 into an existing brief is illustrated in FIG. 6. As illustrated in FIG. 6, the tag 200 may be placed on an inside surface of the sanitary product. For example, on an absorbent core of the sanitary product. However, any placement is contemplated and may depend on a desired purpose of the sensor.

However, in other embodiments, the eBrief 102 may be formed such that the sanitary product and the sensor are a unitary device and assembly may not be required.

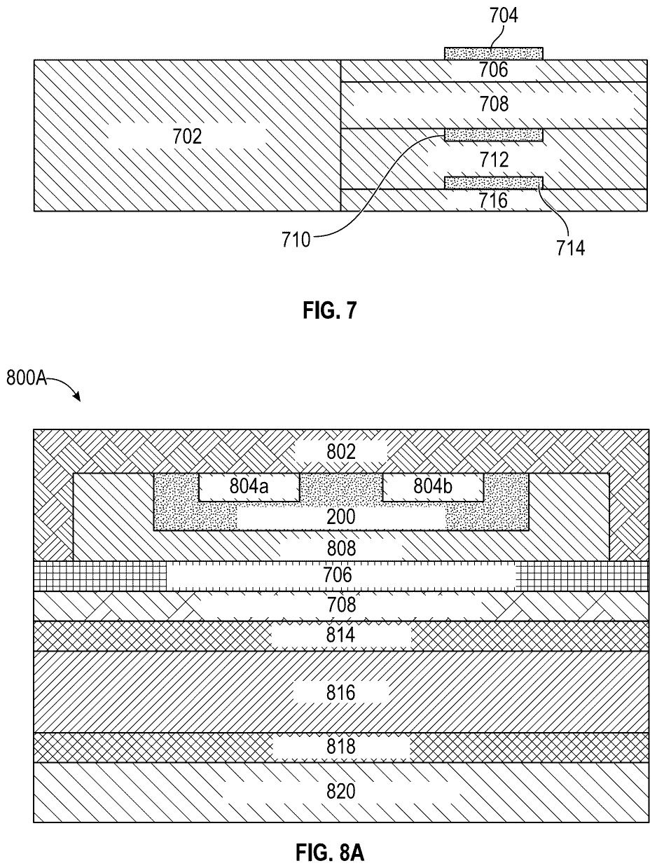

FIG. 7 illustrates three exemplary placements of the tag 200 within the sanitary product. In some embodiments, the tag 200 may be positioned at one of the exemplary placement locations. However, in other embodiments, the eBrief 102 may comprise a plurality of tags 200 that may be placed at any of the exemplary or other placements within the brief.

FIGS. 8A-C illustrate various embodiments of the sensor in first tag location 704 illustrated in FIG. 7. The sticker embodiment of the tag 200 may be utilized to secure the tag 200 to a top portion of the sanitary product. In the embodiment illustrated in FIG. 8A, the tag 200 may be placed “face-up” such that its electrodes are facing the wearer's skin. As illustrated, the electrodes may face a non-woven material 802 that is secured to the brief by an adhesive 808, such as tape.

In the embodiment illustrated in FIG. 8B, the tag 200 may be placed in a “face-down” position such that its electrodes are facing away from the wearer. The tag 200 may be secured to the sanitary product by an adhesive 808, such as tape.

It is contemplated that the “face-up” and “face-down” configurations disclosed in FIGS. 8A-B may determine a sensitivity of the sensor. As a result, the sensor may activate at varying levels of moisture.

In some embodiments, the tag 200 may comprise an absorbent layer positioned between the electrode and the surface it is facing. For example, as illustrated in FIG. 9, the absorbent layer may be positioned between the non-woven material 802 and the electrode.

FIG. 10 illustrates one embodiment of the tag 200 positioned at second tag location 710 from FIG. 7. More particularly, the tag 200 in FIG. 10 is illustrated as being positioned in a core of the sanitary product. In this embodiment, the tag 200 is placed below the topsheet 706 and acquisition distribution layer 708, in contact with the absorbent core 712 of the brief. This allows the tag 200 to activate only when the absorbent core 712 absorbs sufficient fluid to wet the surface of the tag 200, thereby reducing activation at small void volumes.

FIGS. 11A-B illustrate a cross-section schematic diagram showing possible tag 200 placement locations in the layer architecture of an example sanitary product at third tag location 714 of FIG. 7. As illustrated, in this embodiment, the tag 200 is attached to the backside of the absorbent core 712. Similar to the embodiment illustrated in FIG. 10, the absorbent core 712 may reduce the detection of moisture. For example, in FIGS. 11A-B the sensor may only activate when the absorbent core 712 is near full capacity.

In some embodiments, the system may comprise a method of reducing sensitivity of the tag 200 to volumes of fluid. For example, in an embodiment, the tag 200 may be a moisture sensing tag which only activates upon a pre-determined voiding volume. As a result, small voids will not activate the tag 200 and thereby will not create false alarms.

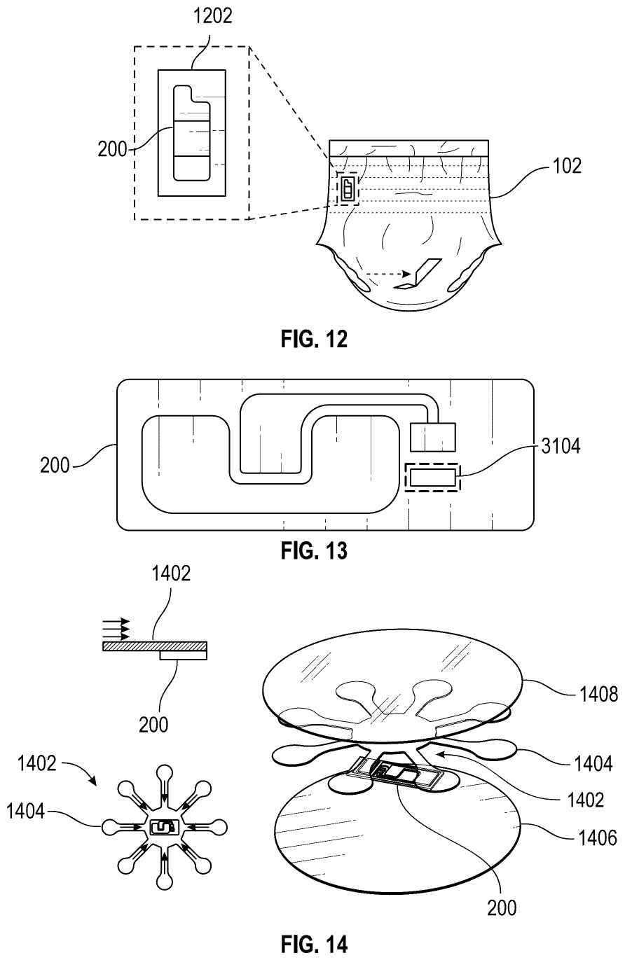

In the embodiment illustrated in FIG. 12, a tag 200 comprising a water soluble film 1202 disposed over its electrodes is illustrated. The water soluble film 1202 may be configured to dissolve after exposure to sufficient fluid and/or time, permitting access to the electrodes. It is contemplated that the water soluble film 1202 of FIG. 12 may reduce the probability of a small/insignificant void activating the sensor. Instead, sufficient fluid volume must be voided in order to dissolve the water soluble film 1202, exposing the electrodes, and activating the tag 200.

In the embodiment of the tag 200 illustrated in FIG. 13, the tag 200 comprises a capacitor 210 to increase the liquid exposure time and/or volume required to activate the tag 200. In some embodiments, the capacitor 210 must charge to a sufficient voltage prior to achieving activation voltage of the tag 200. This charging effect is contemplated to delay the onset of the signal by taking additional time to increase circuit voltage to the necessary voltage. If circuit voltage is not increased to a minimum value fast enough, then the absorbency of the hygiene product will draw fluid away from the sensor before it can activate. The tag 200 may be activated once sufficient fluid is in contact with the sensor and the capacitor 210 charging time can be achieved.

FIG. 14 illustrates one embodiment of reducing the sensitivity of the tag 200 comprising a lateral flow membrane 1402 used to delay the onset of void signal. The lateral flow membrane 1402 may kinetically limit fluid transport to the tag 200 surface. The porosity and/or hydrophobicity of the membrane may be adjusted such that a quantity of moisture greater than a threshold is required to activate the sensor. In some embodiments, the lateral flow membrane 1402 may be oriented in a pattern which extends to various parts of the brief to monitor other endpoints, such as bowel movements, or may be tuned to only pass specific metabolites that may be monitored by the system to indicate potential health risks, such as dehydration, controlled substances, medications, and/or urinary tract infections (UTI). Of course, the lateral flow membrane 1402 may be configured for various purposes and the aforementioned are provided as non-limiting examples only.

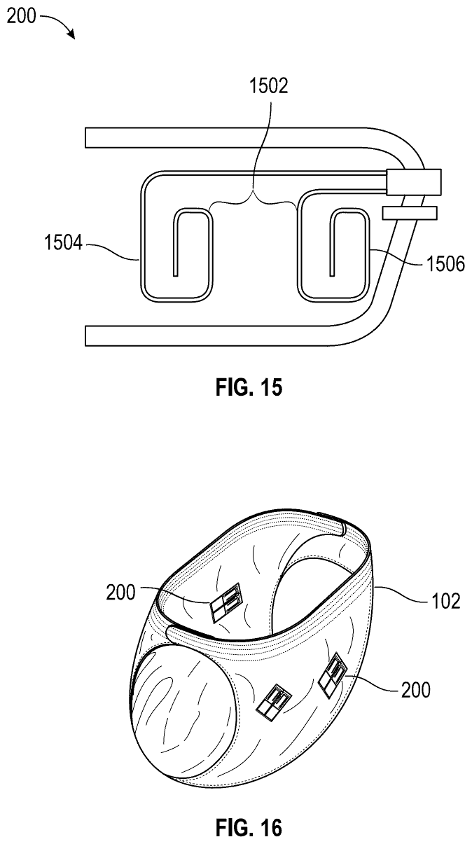

FIG. 15 illustrates one embodiment of a tag 200 comprising a large electrode spacing 1502. It is contemplated that the large electrode spacing 1502 may require a large area of saturation of the sanitary product to complete the electric circuit. In one embodiment, the tag 200 may comprise a galvanic cell with electrode spacing at a distance such that a minimum fluid volume is required to saturate the sanitary product in order to wet both first electrode 1504 and second electrode 1506 and activate the tag 200. Insufficient fluid volume will not bridge both electrodes, and thus, will not activate the tag 200.

In the embodiment illustrated in FIG. 16, the eBrief 102 may comprise a plurality of tags 200 used to monitor the level of saturation of a sanitary product. In such an embodiment, the system will only determine a voiding event when a pre-determined number of tags 200 are active in the sanitary product at the same time. It is contemplated, that in some embodiments, the tag 200 may be further used to differentiate between bowel movements (BM) and urinary voids.

FIG. 17 illustrates an embodiment of an eBrief 102 comprising the tag 200 placement discussed with reference to FIGS. 11A-B. In this embodiment, the sanitary product may serve as the activation moisture threshold. Insufficient quantities of fluid may be absorbed by the sanitary product. Once the sanitary product is sufficiently saturated, the fluid in the absorbent layers of the sanitary product may contact, and thus activate, the tag 200.

Another embodiment of creating a moisture detection threshold is illustrated in FIG. 18. As illustrated the tag 200 may be a skin-contacting tag configured to monitor moisture at the skin 1702 surface. In this embodiment, the tag 200 may be placed in contact with the skin 1702, such that the tag 200 only activates when the skin 1702 itself becomes wet. In some such embodiments, a galvanic cell may be extended such that it proves the sanitary product's absorbent core. The electronic circuit may remain on the outside or inside layers of the brief.

FIG. 19 illustrates a further embodiment of a moisture detection threshold means comprising a super absorbent polymer (SAP) layer to retain fluid on the tag 200 surface once wet.

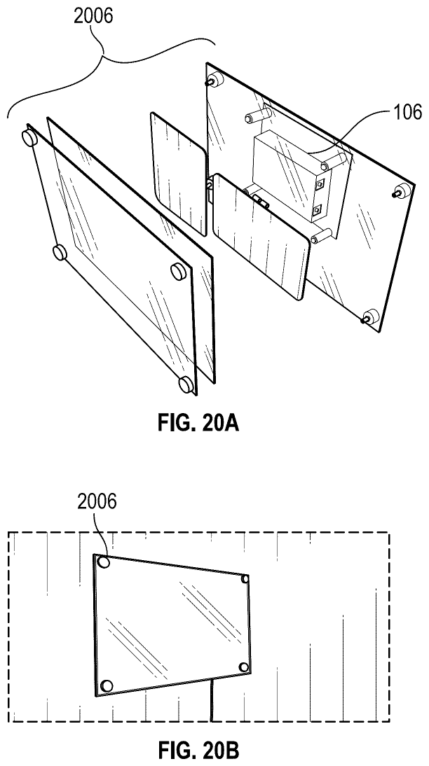

The sensor may be in electronic communication with the reader 106 to detect the presence of moisture. In one embodiment, the reader 106 may be a room reader that is configured to receive communications from sensors in the room. In some embodiments, the reader 106 may be wall-mounted. For example, in an embodiment, the reader 106 may be concealed in common wall-mounted items such as artwork 2006 as shown in FIG. 20.

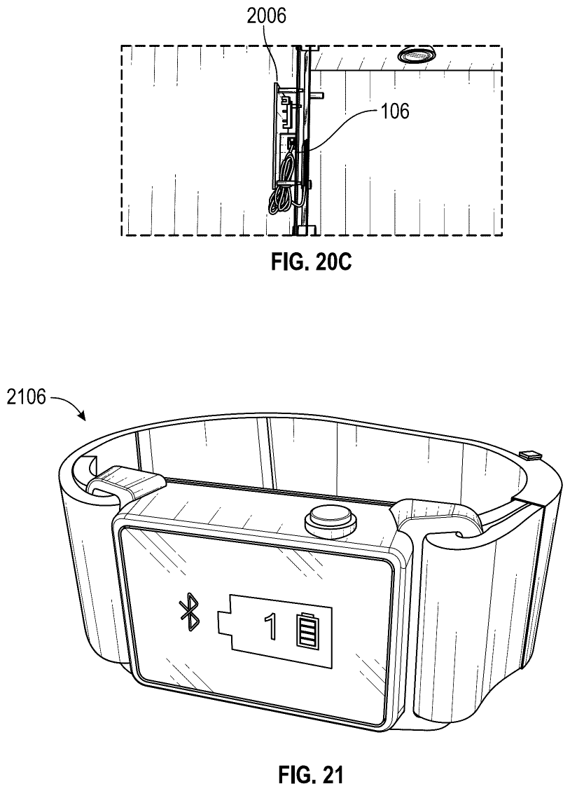

FIG. 21 illustrates an embodiment of a wearable reader 2106 configured as a portable, wearable device. It is contemplated that such an embodiment can detect the presence of moisture in a wide variety of environments. For example, when the wearer has traveled outside of the facility.

In some embodiments, the system may comprise a smart bin. It is contemplated that the smart bin may streamline ease-of-use and may provide additional health monitoring data for caretakers. In an embodiment, the smart bin may be equipped with electronics that detect signals from the sensors, which when integrated into sanitary products, can accurately detect voids when they occur and alert caretakers of a required change. Once voided sanitary products are removed and placed into the bin, void-related attributes are reported to improve and maintain the wearer's health.

In some embodiments, the reader 106 can receive a signal from the sensor from a range of up to 60 feet away. Of course, the signal range may vary, and any range is contemplated.

In the embodiment illustrated in FIG. 22, the smart bin may display the bin capacity status. Further, in some embodiments, the smart bin may automatically log eBrief 102 information, such as change events, times, and volumes of the void. Logging the eBrief 102 information is contemplated to provide continuous monitoring for dehydration, irregular voiding and/or changing patterns, and UTI detection. Further, when integrated with the point of care unit 108 to provide any of the eBrief 102 information to a user. Additionally, in some embodiments, the point of care unit 108 may generate meaningful void pattern statistics, such as average void volume, frequency, and/or time wet.

One embodiment of a point of care unit 108 is illustrated in FIG. 23. However, in other embodiments the point of care unit 108 may be a mobile device, laptop computer, tablet, or any other electronic device. The point of care unit 108 may access a caretake application, webpage, or other interface to interact with the system. The point of care unit 108 may be configured to notify a user, such as a caretaker, when a wearer has voided. In some embodiments, the point of care unit 108 may be further configured to assist with regular rounding by indicating the time since a resident was last changed, track toileting events, and/or track rounding checks. In an embodiment, the point of care unit 108 may determine whether irregular voiding patterns occur with the wearer and predict whether a wearer may have some other underlying health condition, such as a medication reaction, blood sugar issue, UTI, or dehydration.

It is contemplated that voiding patterns may also be used by the facility to manage brief inventories, ordering, and supply chain, thereby reducing operating costs. For example, in some embodiments, the reader 106 and the point of care unit 108 may be further configured to track inventory. For example, the reader 106 may communicate with the point of care unit 108 to notify users of low eBrief 102 inventory. In an embodiment, the reader 106 may report eBrief 102 inventory and usage trends. In a further embodiment, the system may auto-order eBrief 102 supplies when required.

FIG. 24 illustrates one embodiment of a reader-assigned workflow 2400 for the system utilizing the reader 106. In this embodiment, a reading device within a resident's room detects a void event. Each room reader may be assigned to a resident and has a unique ID. It is contemplated that this embodiment may be primarily used in immobilized or independent living/home-based users where the risk of residents entering other rooms while actively voiding is minimal.

In this embodiment, a caretaker receives an alert on a dashboard displayed at the point of care unit 108 identifying which resident room is wet. The caretaker locates the resident and the resident's eBriefs 102, removes and disposes the eBrief 102 into a bin which attenuates the signal from the eBrief 102, dons a new eBrief 102 on the resident, and sanitizes. The caretaker then taps the application dashboard to clear the alert to indicate a change was made.

Another embodiment of a workflow for the system is illustrated in FIG. 25 utilizing a Tag-ID. In this embodiment, a batch of eBriefs 102 is assigned to a resident which transmits a unique ID which is stored in a database associated with the caretaker application and/or stored locally or on a server. The unique ID can be detected by any reader 106 in the facility, and the dashboard will accurately indicate which resident has voided regardless of where they are in the facility. As evident in step 2440, the caretaker now must apply the eTag to the standard hygiene product, in this case a brief, prior to donning the brief. This thereby allows the off-the-shelf product to be converted at point of use to a smart “eBrief 102” product, for example, utilizing the workflows disclosed with reference to FIGS. 3-6. All remaining steps may remain the same. Of course, the method may be altered and/or deviated from and use of the system is not limited to any of the disclosed workflows.

In this embodiment, the unique reader ID may also be transmitted to the database so the Application dashboard can show both the resident who voided and the location of the nearest reader 106 which made the detection, thereby providing the location of the resident in the facility.

FIG. 26 illustrates a fully automated scanner assigned workflow 2600 for the system utilizing a scanner assigned workflow. In this embodiment, a scanner detects a new eBrief 102 by various means (such as, but not limited to RFID, BLE, NFC, barcode, or QR). In some embodiments, this may be upon dispensing an eBrief 102 from a device such as a smart dispenser which reads the eBrief's 102 unique ID in digital or machine readable form upon removal of the eBrief 102 from the dispenser, or manually scanned by a hand-held reader, or ambiently detected by a room RFID reader once removed from the dispenser, in which case the dispenser may act as an RF shield preventing the eBrief 102 from being sensed by the system until it is in use. Of course, other means of detecting the new eBrief 102 may be utilized.

Once scanned, the eBrief tags 200 unique ID is assigned to the resident. The association of the eBrief 102 ID and resident are stored in a database. Later, when the resident voids in the eBrief 102 and it signals, the detected signal is compared to the database and the assigned resident is reported as wet on the caretaker dashboard. The resident location to the nearest proximal reader 106 may also be reported so staff can be dispatched to the correct location to find the resident requiring a change. Upon changing the eBrief 102, a disposal bin can be used to hold the soiled eBrief 102 to contain liquids, solids, and odors. In some embodiments, the bin may be the smart bin, however, in other embodiments, the bin may be a traditional bin. The bin, bin liner, or refuse bag, can also be constructed of metal in sheet, woven, screen, or mesh form, to prevent RF signal from emanating from the disposal bin once a soiled eBrief 102 is disposed of into the bin. This will prevent multiple alerts from being generated by the soiled eBrief 102. In other embodiments, the database can be alerted to ignore future signals from this eBrief 102 once a change is made, thereby removing the requirement to shield the soiled eBrief's 102 signal emanating from the bin.

In some embodiments, the scanning process discussed in FIG. 26 may be automated. This embodiment, illustrated in FIG. 27 may automate the scanning function to create a completely auto-assigned workflow 2700. In this embodiment, eBriefs 102 are initialized and assigned to the resident database by various means such as wireless radio (eBurst, RFID, NFC, BLE) or other communications technology once the eBrief 102 is donned on the resident. Once voided, a void event is detected by the sensor and transmits a signal to the reader 106, also communicating the resident identification, such as the resident's room number or name, location, and time of the event to the database by means of a reader 106 or plurality of readers 106 placed within the facility. The caretaker then disposes the resident's soiled eBrief 102. Disposing the eBrief 102 into the smart bin will close the open eBrief 102 by means of a detection system within the eBin 104, notifying the time of change for the resident to the database, and optionally updating the resident's inventory log. The cycle repeats when a new eBrief 102 is pulled from the dispenser.

In a further embodiment, the system may utilize image processing methods. For example, a room may be fitted with optical sensors, cameras, or other, so that optical methods may be employed to detect residents by facial recognition, gait, posture, and/or other items of context in a room such as a hygiene product, staff, or other items of interest. A void event may be detected by use of infrared cameras and detecting a rapid increase in localized body surface temperature at the site of the void by infrared (IR) camera or other wavelengths. A change may be indicated to the system database by using image processing techniques to identify residents, caretakers, and briefs, brief packages, or other hygiene products and their behaviors within a room. In some embodiments, AI/ML methods may be used to monitor behaviors and create predictive models indicative of brief changes. Models may be created and trained to accurately determine when caretakers are changing a resident and/or when a resident has voided.

FIG. 28A, FIG. 28B and FIG. 29 illustrate exemplary embodiments for dataflow and connectivity architectures 2800 according to any of the disclosed embodiments.

The dataflow and connectivity architectures 2800 may include multiple interconnected components that facilitate communication and data exchange within the moisture detection system. In some aspects, the architecture may comprise physical readers 2900, gateway applications 2940, and point-of-care (POC) applications 2850 working in concert to process and relay moisture event data.

Physical readers 2900, which may be distributed throughout a facility, may detect signals from eBrief tags 200 when moisture events occur. These readers 2900 may communicate via Bluetooth or other short-range wireless protocols with gateway applications 2940 running on Android smartphones or similar devices. The gateway applications 2940 may serve as intermediaries, converting detected events into JSON format and associating them with specific physical readers 2900.

The system may utilize a facility's wireless local area network (WLAN) 2835 to enable communication between various components. Gateway applications 2940 may transmit processed event data over WiFi to a central server, which may be referred to as the host server 2805 in some implementations. This server 2805 may handle various functions including inbound JSON message processing, data storage, room and reader association management, and generation of outbound messages.

In some embodiments, the host server 2805 may communicate with the internet 2815 and potentially third-party servers 2825 using JSON/HTTPS protocols. This may allow for integration with external systems or services as needed.

The architecture may also include POC applications 2850 running on various devices such as Android smartphones, tablets, or other mobile devices. These applications 2850 may interface with the system to display care event information, allow user input, and facilitate brief or resident management tasks. In some cases, POC apps 2850 may be configured for specific rooms or groups of rooms, providing targeted information to caregivers.

A dashboard component 2860 may be included in the architecture, potentially running on a PC or other device with a web browser. This dashboard 2860 may provide administrative functions, enrollment capabilities, and real-time monitoring of events across the system.

The dataflow within this architecture 2800 may generally follow a path from physical readers 2900 detecting events, through gateway apps 2940 for initial processing, to the central server 2805 for data management and storage, and finally to POC apps 2850 and dashboards 2860 for user interaction and monitoring. This structure may allow for efficient handling of moisture events, from initial detection to caregiver notification and response.

A tag/sensor 200 assembly process is described herein. In one embodiment, the assembly process may incorporate magnesium into the tag 200 by means of stamping or placing a magnesium thin film, printing a custom magnesium metallic ink or nano-ink, or sputtering magnesium onto the surface of the material using a mask. One embodiment of a tag 200 constructed according to the process is illustrated in FIG. 2.

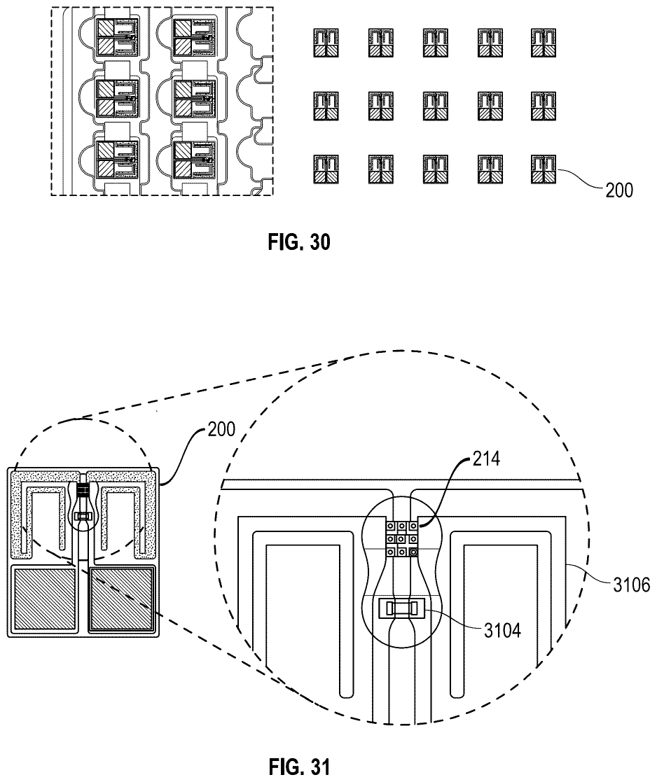

FIG. 30 illustrates a plurality of tags 200 constructed according to the assembly process. In some embodiments, the plurality of tags 200 may comprise various tag IDs 3106. The tag IDs 3106 may be controlled by layout during the manufacturing of the copper layer pattern. Further, in some embodiments, the tag IDs 3106 may be labeled for easy identification of individual tags 200.

FIG. 31 shows a detailed engineering drawing view of an embodiment of the tag 200 showing the integrated circuit 214, capacitor 210 attachment, and tag ID 3106 identifier metal patterns.

FIG. 32 illustrates various manufacturing process workflows of the assembly process. Of course, the workflows illustrated in FIG. 32 are provided as non-limiting examples and any method of manufacturing may be utilized.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claim.

A number of implementations have been described. Nevertheless, it will be understood that various modifications may be made without departing from the spirit and scope of the disclosure. Accordingly, other implementations are within the scope of the following claims.

1. A system for monitoring incontinence care, comprising:
a plurality of hygiene products or undergarments, each hygiene product or undergarment having an associated tag;
a reader configured to detect the tags associated with the hygiene products or undergarments;
a point of care unit configured to receive tracking information from the reader, wherein the point of care unit is further configured to track any of the time since a resident was last attended to, moisture events, and care events, wherein care events comprise checking the resident, changing the resident's hygiene product or undergarment, or toileting the resident, and wherein the point of care unit is configured to generate an alert when a resident has been unattended for longer than a predetermined time period.

2. The system of claim 1, wherein the tags comprise radio frequency identification (RFID) tags configured to be detected by the reader within a specified range.

3. The system of claim 1, wherein the point of care unit comprises a display configured to show a status icon indicating a wet status or a dry status for each resident.

4. The system of claim 3, wherein the display of the point of care unit further indicates a room or bed for each resident.

5. The system of claim 3, wherein the status icon further indicates an elapsed time since at least one of:
a most recent moisture event detected for each of the plurality of hygiene products or undergarments, or
a most recent care event performed for each resident associated with the plurality of hygiene products or undergarments.

6. The system of claim 1, further comprising a smart bin configured to detect disposal of soiled hygiene products or undergarments.

7. The system of claim 6, wherein the smart bin is further configured to:
detect a fluid volume of waste in the soiled hygiene products or undergarments; and
determine, based on the fluid volume of waste, a hydration state of the resident.

8. A sensor insert for a hygiene product, comprising:
a non-woven material forming an uppermost layer;
a super absorbent polymer layer positioned below the non-woven material;
a tag positioned below the super absorbent polymer layer, the tag configured to detect moisture, wherein the super absorbent polymer layer is configured to consolidate the moisture at the tag;
an inner adhesive layer positioned below the tag; and
a back sheet positioned below the inner adhesive layer, wherein the sensor insert is configured to be adhered to an inside surface of the hygiene product.

9. The sensor insert of claim 8, further comprising an outer adhesive layer positioned below the back sheet.

10. The sensor insert of claim 9, further comprising a release liner removably attached to the outer adhesive layer.

11. The sensor insert of claim 8, wherein the sensor insert further comprises an inner adhesive layer configured to secure the tag between the super absorbent polymer layer and the back sheet.

12. The sensor insert of claim 8, wherein the tag comprises a first electrode and a second electrode, wherein the tag is configured to detect moisture when the moisture bridges the first electrode and the second electrode.

13. The sensor insert of claim 8, wherein the sensor insert is configured to be positioned in a first location for a male patient and a second location for a female patient.

14. The sensor insert of claim 13, wherein the first location is toward a front portion of the hygiene product and the second location is toward a middle portion of the hygiene product.

15. A method for handling refuse or hygiene product disposal in a bin, comprising:
receiving a hygiene product in a bin;
determining the hygiene product is a soiled hygiene product;
measuring a fluid volume of waste in the soiled hygiene product and displaying a message indicating an event recorded for a resident;
determining, at least partially based on the fluid volume of waste, a hydration level of the resident.

16. The method of claim 15, further comprising tracking a fluid intake of the resident.

17. The method of claim 16, wherein the determination of the hydration level of the resident is further based on the fluid intake of the resident.

18. The method of claim 15, wherein determining whether the hygiene product is the soiled hygiene product comprises detecting whether the hygiene product transmits a signal.

19. The method of claim 18, wherein detecting the expired hygiene product comprises:
detecting a weight increase in the bin; and
determining that no signal is received from the hygiene product in the bin.

20. The method of claim 19, further comprising: after detecting the soiled hygiene product, providing an option for a user to confirm the detection was accurately recorded.
